(19) United States
(12) Patent Application Publication
(10) Pub. No.: US 2020/0338128 A1
(43) Pub. Date: Oct. 29, 2020

(54) PRECISION MOLECULAR ADAPTOR SYSTEM FOR CAR-T IMMUNOTHERAPY

(71) Applicant: Gencyte Therapeutics, Inc., San Jose, CA (US)

(72) Inventor: Chuhua Zhong, San Jose, CA (US)

(73) Assignee: Gencyte Therapeutics, Inc., San Jose, CA (US)

(21) Appl. No.: 16/958,915

(22) PCT Filed: Jan. 7, 2019
(86) PCT No.: PCT/US2019/012468

§ 371 Date: Jun. 29, 2020

Related U.S. Application Data
(60) Provisional application No. 62/614,000, filed on Jan. 5, 2018.
(60) Provisional application No. 62/643,077, filed on Mar. 14, 2018.

(51) Int. Cl. A61K 35/17; A61K 49/00; C07K 16/28; C12N 15/85; C07K 14/725

(52) U.S. Cl. CPC A61K 35/17; A61K 49/0034; A61K 49/0058; C07K 2317/622; C12N 15/85; C07K 14/7051; C07K 2319/03; C07K 16/2803

(57) ABSTRACT

The invention provides compositions and methods for immunotherapy including a modular CAR (mCAR) and a precision molecular adaptor (PMA) for targeting the modular CAR-T cells to one or more tumor antigen(s).

According to one embodiment of the invention, a precision molecular adaptor (PMA) is provided that comprises: (a) a recognition moiety comprising an indocyanine green (ICG) moiety, and, attached to the recognition moiety, (b) an antigen-binding moiety comprising a first antigen recognition domain that binds specifically to a first target antigen. In fact, the antigen-binding moiety of the PMA can be multi-specific, binding to two, three, or more target antigens. According to one embodiment, the antigen-binding moiety of the PMA is bispecific, further comprising a second antigen recognition domain that binds specifically to a second target antigen that is different than the first target antigen. For example and without limitation, the first and second target antigens may be cell surface proteins or cell surface protein complexes, e.g., an antigen selected from the group consisting of: alpha-fetoprotein, α-actinin-4, A3, antigen specific for A33 antibody, ART-4, B7, Ba 733, BAGE, BrE3-antigen, a member of the carbohydrate antigen family, a member of the carbonic anhydrase family, CA125, CAMEL, CAP-1, CASP-8/m, CCL19, CCL21, CD1, CD1a, CD2, CD3, CD4, CD5, CD8, CD10, CD11A, CD14, CD15, CD16, CD18, CD19, CD20, CD21, CD22, CD23, CD25, CD29, CD30, CD32b, CD33, CD37, CD38, CD40, CD40L, CD44, CD45, CD46, CD52, CD54, CD55, CD59, CD64, CD66a-e, CD67, CD70, CD70L, CD74, CD79a, CD79b, CD80, CD83, CD95, CD99, CD 123, CD126, CD132, CD133, CD138, CD147, CD154, CD274, CDC27, CDK-4/m, CDKN2A, CTLA4, CXCR4, CXCR7, CXCL12, HIF-1α, colon-specific antigen-p, CEACAM-5, CEACAM-6, c-Met, DAM, a diasialoganglioside, an embryonic antigen, a member of the epidermal growth factor receptor family and mutants thereof, a member of the ephithelia glycoprotein family, EGP-1, EGP-2, ELF2-M, Ep-CAM, a member of the ephrin receptor family, erb-1, erb-2, fibroblast growth factor, Flt-1, Flt-3, folatebinding protein, α-folate receptor, follicle stimulating hormone receptor, G250 antigen, GAGE, gp100, a member of the Rho family of GTPases, GRO-0, HLA-DR, HM1.24, human chorionic gonadotropin and its subunits, HER2/neu, a member of the high mobility group proteins and mutants thereof, HMGB-1, human high molecular weight-melanoma-associated antigen, hypoxia inducible factor, HSP70-2M, HST-2, Ia, IGF-1R, IFN-γ, IFN-α, IFN-β, IFN-k, IL-4R, IL-6R, IL-13R, IL-15R, IL-17R, IL-18R, IL-2, IL-6, IL-8, IL-12, IL-15, IL-17, IL-18, IL-23, IL-25, insulin-like growth factor-1, KC4-antigen, KS-1-antigen, KS1-4, Le-Y, LDR/FUT, a ligand of the NKG2D receptor, macrophage migration inhibitory factor, MAGE, MAGE-3, MART-1, MART-2, NY-ESO-1, TRAG-3, mCRP, MCP-1, MIP-1A, MIP-1B, MIF, a members of the mucin protein family, MUM-1/2, MUM-3, NCA66, NCA95, NCA90, pancreatic cancer mucin, PD1 receptor, placental growth factor, p53, PLAGL2, prostatic acid phosphatase, a prostrate-specific antigen, PRAIVIE, PlGF, ILGF, ILGF-R, IL-6, IL-25, RS5, RANTES, T101, SAGE, S100, survivin, survivin-2B, TAC, TAG-72, tenascin, TIM-3, a TRAIL receptor, TNF-α, Tn antigen, a Thomson-Friedenreich antigen, a tumor necrosis antigen, a members of the vascular endothelial growth factor receptor family, ED-B fibronectin, WT-1, 17-1A-antigen, complement factor C3, complement factor C3a, complement factor C3b, complement factor C5a, complement factor C5, an angiogenesis marker, bcl-2, bcl-6, Kras, an oncogene marker and an oncogene product. According to one embodiment, the first and second target antigens are CD19 and CD22, respectively. As one example, the antigen-binding moiety may comprise a single-chain variable fragment (scFv).

According to another embodiment of the invention, methods are provided for making a PMA comprising: providing an antigen-binding moiety comprising a first antigen recognition domain that binds specifically to a first target antigen, and attaching an indocyanine green (ICG) moiety to the antigen-binding moiety.

According to another embodiment of the invention, a chimeric antigen receptor (CAR) is provided that comprises: (a) a binding domain that specifically binds to indocyanine green (ICG); (b) an extracellular hinge and transmembrane domain; and (c) a signal transduction domain. For example, the binding domain may comprise an antibody or antibody fragment that specifically binds to ICG, e.g., an scFv. In such an embodiment, the signal transduction domain may comprise a T cell activation signal region; the signal transduction domain may further comprise one or more costimulatory signal regions. According to another embodiment, a polynucleotide construct, or vector, is provided comprising a promoter operably linked to a sequence that encodes such a CAR. According to another embodiment, an effector cell comprising such a polynucleotide construct is provided. Such effector cells include, without limitation: lymphoid lineage cells, e.g., T cells Natural Killer (NK) cells, cytotoxic T lymphocytes (CTLs), or regulatory T cells; or myeloid lineage cells, e.g., neutrophils or macrophages; or other cells such as rδ T cells, Natural Killer T cells (NKT cells) or lymphokine-activated killer (LAK) cells. Such cells may be autologous cells (i.e., cells harvested from a patient and returned to the patient after introduction of a CAR vector into the cells) or heterologous cells, e.g., allogeneic cells.

According to another embodiment of the invention, a two component therapeutic is provided that comprises: (1) a composition comprising a plurality of effector cells that comprise a vector comprising a eukaryotic promoter operably linked to a sequence that encodes a chimeric antigen receptor (CAR), the CAR comprising (a) a binding domain that specifically binds to indocyanine green (ICG); (b) an extracellular hinge and transmembrane domain; and (c) a signal transduction domain; and (2) a composition comprising a precision molecular adaptor (PMA), the PMA comprising: (a) a recognition moiety comprising an indocyanine green (ICG) moiety, and, attached to the recognition moiety, (b) an antigen-binding moiety comprising a first antigen recognition domain that binds specifically to a first target antigen.

According to another embodiment of the invention, a method of treating a mammal having a disease is provided, the method comprising: introducing into the mammal (e.g., a human) a therapeutically effective amount of a composition comprising one or more effector cells that comprise a vector comprising a eukaryotic promoter operably linked to a sequence that encodes a chimeric antigen receptor (CAR), wherein the CAR comprises: (a) a binding domain that specifically binds to indocyanine green (ICG); (b) an extracellular hinge and transmembrane domain; and (c) a signal transduction domain; and introducing into the mammal a therapeutically effective amount of a composition comprising a precision molecular adaptor (PMA), the PMA comprising: (a) a recognition moiety comprising an indocyanine green (ICG) moiety, and, attached to the recognition moiety, (b) an antigen-binding moiety comprising a first antigen recognition domain that binds specifically to a first target antigen; thereby reducing a symptom of the disease.

According to another embodiment, the foregoing methods may also comprise introducing into one or more effector cells from the mammal said vector, thereby producing said plurality of cells that comprise said vector. According to another embodiment, the foregoing methods may also comprise isolating said effector cells from the mammal, and introducing the vector into the cells.

According to another embodiment of the invention, methods are provided for identifying locations of cells comprising a CAR, the method comprising: (1) introducing into the mammal a therapeutically effective amount of a composition comprising one or more cells that comprise a vector comprising a eukaryotic promoter operably linked to a sequence that encodes a chimeric antigen receptor (CAR), wherein the CAR comprises: (a) a binding domain that specifically binds to indocyanine green (ICG); (b) an extracellular hinge and transmembrane domain; and (c) a signal transduction domain; (2) introducing into the mammal a therapeutically effective amount of a composition comprising a precision molecular adaptor (PMA), the PMA comprising: (a) a recognition moiety comprising an indocyanine green (ICG) moiety, and, attached to the recognition moiety, (b) an antigen-binding moiety comprising a first antigen recognition domain that binds specifically to a first target antigen; and (3) performing near infrared fluorescent imaging to identify the location of the cells.

According to another embodiment of the invention, kits are provided for producing a precision molecular adaptor (PMA), the kits comprising: a first component comprising an indocyanine green (ICG) moiety, a second component comprising an antigen-binding moiety comprising a first antigen recognition domain that binds specifically to a first target antigen, and a container for said first and second components, wherein the PMA comprises said first component attached to said second component. According to another embodiment of the invention, kits are provided that comprise: a first component selected from the group consisting of: a vector comprising a eukaryotic promoter operably linked to a sequence that encodes a chimeric antigen receptor (CAR), the CAR comprising a binding domain that specifically binds to indocyanine green (ICG), an extracellular hinge and transmembrane domain, and a signal transduction domain; and composition comprising an effector cell comprising said vector; and a second component consisting of a composition comprising a precision molecular adaptor (PMA), the PMA comprising a recognition moiety comprising an indocyanine green (ICG) moiety, and, attached to the recognition moiety, an antigen-binding moiety comprising a first antigen recognition domain that binds specifically to a first target antigen; and a container for said first and second components.

### BRIEF DESCRIPTION OF THE DRAWINGS

FIG. 1A shows the structure of an mCAR DNA construct.

FIG. 1B shows a lentiviral vector for introducing mCARs into T cells.

FIG. 2 is a schematic diagram of the PMA-mCAR-T system

FIG. 3 illustrates dual targeting to prevent tumor antigen escape (A=CD19, B=CD22).

FIG. 4 is a schematic diagram of a monospecific MA-mCAR-T system.

### DETAILED DESCRIPTION OF THE INVENTION

### Overview

The present invention provides compositions and methods for CAR immunotherapy. The CAR-T platform of the present invention includes two parts: (1) a modular CAR (mCAR) that is engineered for reduced toxicity and immunogenicity to human cells; and (2) a precision molecular adaptor (PMA) for targeting the modular CAR-T cells to specific tumor antigen(s). The PMA includes a fixed recognition moiety that is bound by the binding region of mCAR, and a modifiable targeting moiety for binding to two specific tumor antigens.

The construction and use of CAR and CAR-T were reviewed by Sadelain et al. (Cancer Discov 3:388-398, 2013).

First generation CARs comprised two main regions. First, a recognition region, e.g., a single chain fragment variable (scFv) region derived from a tumor-targeted antibody, is used to recognize and bind tumor-associated antigens. Second, an activation signaling domain, e.g., the CD3 transmembrane domain, with an intracellular CD3- or FcRy endodomain, serves as a T cell activation signal (Cartellieri et al., J. Biomedicine and Biotechnology Vol. 2010, Article ID 956304, 2010). Although T cells transduced to express such constructs showed positive results in vitro, they were been found to have limited performance in eliminating tumor cells in clinical trials. The main limitation was the relative inability to prolong and expand the T cell population and achieve sustained antitumor effects in vivo (Sadelain et al., Cancer Discov 3:388-398, 2013).

To address these problems, second generation CARs provide a dual signaling function to combine T-cell activation with costimulatory signals, such as cytokine (e.g., IL-2, IL-7, IL-15, IL-21) release. The second generation constructs comprised CD28 or CD3-ζ transmembrane domains, attached to two or more intracellular effectors selected from CD28 endodomain, CD3-ζ endodomain, ICOS, 4-1BB, DAP10 and OX40. The addition of a co-stimulation domain enhances the in vivo proliferation and survival of T cells containing CARs (Sadelain et al., 2013).

Third generation CARs comprise three or more signaling functions, typically incorporating CD28 transmembrane and endodomains, attached to the signaling subunits of 4-1BB, OX-40 or Lck, and the cytoplasmic domain of CD3-.

CAR constructs also have been used to direct natural killer (NK) cell activity (reviewed by Hermanson and Kaufman, Front Immunol 6:195, 2015; and Carlsten and Childs, Front Immunol 6:266, 2015). Like T cells, NK cells can be transfected with CAR expression constructs and used to induce an immune response. Because NK cells do not require HLA matching, they can be used as allogeneic effector cells (Harmanson & Kaufman, 2015). Also, peripheral blood NK cells (PB-NK) may be isolated from donors by a simple blood draw.

CAR constructs for use in NK cells (CAR-NK) may contain similar elements to those used to make CAR-T cells. CAR-NK cells may contain a targeting molecule, such as a scFV or Fab, that binds to a disease associated antigen, such as a tumor-associated antigen (TAA), or to a hapten on a targetable construct. This avoids the problem that NK cells, unlike T cells, lack antigen specificity for targeting cells to be killed. The cell-targeting scFv or Fab may be linked via a transmembrane domain to one or more intracellular signaling domains to effect lymphocyte activation. Signaling domains used with CAR-NK cells have included CD3-ζ, CD28, 4-1BB, DAP10 and OX40. NK cell lines of use have included NK-92, NKG, YT, NK-YS, HANK-1, YTS and NKL cells.

Nucleotide sequences encoding the cDNA of CAR constructs are incorporated in an expression vector, such as a retroviral or lentiviral vector, for transfer into T cells or NK cells. Following infection, transfection, lipofection or alternative means of introducing the vector into the host cell (CAR-T or CAR-NK), the cells are administered to a subject to induce an immune response against antigen-expressing target cells. Binding of CARs on the surface of transduced T cells or NK cells to antigens expressed by a target cells activates the T or NK cell. Activation of T or NK cells by CARs does not require antigen processing and presentation by the HLA system.

A variety of CAR-T or CAR-NK cells have been used for therapy of disease states, primarily hematopoietic cancers or some solid tumors. Targeted antigens have included α-folate receptor (ovarian and epithelial cancers), CAIX (renal carcinoma), CD19 (B-cell malignancies, CLL, ALL), CD20 (B-cell malignancies, lymphomas), CD22 (B-cell malignancies), CD23 (CLL), CD24 (pancreatic CA), CD30 (lymphomas), CD33 (AML), CD38 (NHL), CD44v7/8 (cervical CA), CEA (colorectal CA), EGFRvIII (glioblastoma), EGP-2 (multiple malignancies), EGP-40 (colorectal CA), EphA2 (glioblastoma), Erb-B2 (breast, prostate, colon CA), FBP (ovarian CA), GD2 (neuroblastoma, melanoma), GD3 (melanoma), HER2 (pancreatic CA, ovarian CA, glioblastoma, osteosarcoma), HMW-MAA (melanoma), IL-11Ra (osteosarcoma), IL-13Rα2 (glioma, glioblastoma), KDR (tumor vasculature), κ-light chain (B-cell malignancies), Lewis Y (various carcinomas), L1 (neuroblastoma), MAGE-A1 (melanoma), mesothelin (mesothelioma), MUC1 (breast and ovarian CA), MUC16 (ovarian CA), NKG2D (myeloma, ovarian CA), NY-ESO-1 (multiple myeloma), oncofetal antigen (various tumors), PSCA (prostate CA), PSMA (prostate CA), ROR1 (B-CLL), TAG-72 (adenocarcinomas), and VEGF-R2 (tumor neovasculature). (Sadelain et al., Cancer Discov 3:388-98, 2013).

There remain several challenges to overcome in order to achieve significant clinical outcomes. A first problem is tumor antigen escape. Since the introduction of CD19-based immunotherapies, relapse with diminished or absent cell-surface CD19 has been increasingly observed and has emerged as the dominant mechanism of resistance to this class of therapeutics (Maude et al., N Engl J Med 371:1507-1517, 2014; Grupp et al., Blood. 126:681, 2015). Currently used conventional CARs have single-target specificity. Such a monospecific targeting approach risks the development of tumor escape variants (Anurathapan et al., Mol Ther 22:623-633, 2014; Schneider et al., J ImmunoTherapy Cancer 5:42-59, 2017). Despite high initial response rates, some patients relapse, and up to 60% of relapses after CART19 therapy are characterized by the loss of CD19 antigen, rendering the malignant cells invisible to CD19-specific immunotherapies (Maude et al., N Engl J Med 371:1507-1517, 2014; Anurathapan et al., Mol Ther 22:623-633, 2014; Grupp et al., Blood. 126:681, 2015).

A second problem is “off-target” toxicities, which may result due to difficulty in targeting only cancer cells via tumor-associated antigens, since in many cases normal cells also express the tumor-associated antigen. For example, CD19 is a tumor-associated antigen that is expressed on malignant B cells. CARs containing anti-CD19 antibody were generated and used treated to patients. In one trial, although CAR-T therapy resulted in remission of malignant B cells, normal B cells were depleted in the patients as well because normal B cells also express CD19 (Porter et al., N. Engl. J. Med. 365:725-733, 2011). Another example pertains to carbonic anhydrase IX (CAIX) which is overexpressed in clear cell renal carcinoma. Liver toxicity was found in subjects of the first clinical trial using CAR-targeting CAIX, likely due to the fact that CAIX is also expressed in bile duct epithelial cells and as such, the T cells targeted normal tissue as well (Cor et al., J. Clin. Oncol. 24:e20-22, 2006).

A third problem is cytokine release syndrome (CRS). The fixed antigen-binding moiety on CAR-modified T cells provides no means of direct control over ongoing CAR T-cell reactivity. Leukemia and lymphoma patients treated with CD19-specific CAR-T cells have suffered severe CRS owing to the rapid activation and expansion of CAR-T cells upon encountering CD19-positive cells. After infusion, CAR T cells expand in response to their antigen by more than 1,000-fold (Grupp et al., N Engl J Med. 368:1509-1518, 2013), resulting in the uncontrollable release of cytokines (CRS) from synchronously activated and rapidly proliferating CAR-T cells. CRS is currently managed with anti-IL-6 receptor antibodies or by suppressing CAR-T-cell activity with corticosteroids (Grupp et al., N Engl J Med 368:1509-1518, 2013).

A fourth problem is lack of flexibility to rapidly and efficiently target new antigens. As no single tumor antigen is expressed by all cancer types, scFv encoded by CAR genes needs to be constructed for each potential tumor antigen. A single CAR construct that can target different antigens would be advantageous, for example, in treating tumor escape variants and heterogeneous tumors expressing distinct tumor antigens.

To overcome these limitations, several research groups have tried to develop soluble intermediary “switch” (adaptor) molecules to regulate CAR-T cells (Tamada et al., Clin Cancer Res 18:6436-6445, 2012; Urbanska et al., Cancer Res 72:1844-1852, 2012; Kudo et al., Cancer Res 74:93-103, 2014; Kim et al., J Am Chem Soc 137:2832-2835, 2015; Ma et al., Proc Natl Acad Sci USA, 113:E450-E458, 2016; Rodgers et al., 113:E459-E468, 2016). These switches are comprised of a tumor-targeting antibody or small-molecule ligand and a second moiety that selectively binds the CAR. CAR-T cell activity is strictly dependent on the formation of a ternary complex between the CAR-T cell, switch, and tumor antigen. Therefore, titration or removal of the switch molecule can control or terminate CAR-T cell response, respectively. In addition, the intercellular switch approach disclosed herein enables the targeting of multiple tumor-associated antigens (TAAs) with a “universal” CAR-T cell. These switchable CAR-T cells are expected to remain in patients after termination of treatment. Examples of switches used in this approach include TAA-specific monoclonal antibodies that elicit antitumor activity from chemically or enzymatically modified antibody-hapten conjugates that redirect anti-hapten CAR-T cells (Tamada et al., Clin Cancer Res 18:6436-6445, 2012; Urbanska et al., Cancer Res 72:1844-1852, 2012; Kim et al., J Am Chem Soc 137:2832-2835, 2015; Ma et al., Proc Natl Acad Sci USA, 113:E450-E458, 2016; Rodgers et al., 113:E459-E468, 2016) or Fc-specific CAR-T cells (Kudo et al., Cancer Res 74:93-103, 2014). The switch concept has been effective in vitro and in vivo to clear leukemia cells as well as to control CAR-T activity.

However, these currently disclosed technologies use FITC (Tamada et al., Clin Cancer Res 18:6436-6445, 2012; Kim et al., J Am Chem Soc 137:2832-2835, 2015; Ma et al., Proc Natl Acad Sci USA, 113:E450-E458, 2016), biotin (Urbanska et al., Cancer Res 72:1844-1852, 2012), or a peptide neo-epitope (PNE) (Rodgers et al., 113:E459-E468, 2016) as an epitope in adaptors recognized by CAR. Furthermore, each of the switch constructs described previously targets only a single tumor antigen. These systems may not work well in human trials due to several factors:

(1) Monospecificity and tumor antigen escape: Many tumors display heterogeneous antigens. With single-target CAR-T treatments, even tumors that initially display a single antigen can undergo tumor antigen escape after initial treatment. The monospecific nature of current technologies makes it difficult to control this process and eliminate variants arising during treatment.

(2) General toxicity: The epitopes used in adaptors recognized by CAR, such as FITC, are not approved for clinical applications and may be toxic in humans.

(3) Off-target toxicity: Some binding moieties may cross-react with endogenous molecules and receptors in human normal tissues (e.g., biotin with biotin binding receptor [Urbanska et al., Cancer Res 72:1844-1852, 2012], or folate with folate receptor [Kim et al., J Am Chem Soc 137:2832-2835, 2015]). CD16 (Fc receptor)-based CAR-T cells bind indiscriminately to therapeutic and naturally occurring antibodies (Kudo et al., Cancer Res 74:93-103, 2014), which makes it possible that any endogenous antibody can activate the CD16-CAR, potentially causing off-target effects.

(4) Immunogenicity: For example, PNEs are derived from non-human proteins and may cause the human body to produce antibodies to clear it and thus inactive the CAR-T cells.

To address the limitations of existing switch technology and to increase the possibility of clinical use, the present invention provides a novel precision molecular adaptor (PMA) system for flexible tumor antigen targeting. A soluble precision molecular adaptor (PMA) that determines the tumor specificity is used to target a tumor antigen; according to an alternative embodiment, the PMA is multi-specific, e.g., bi-specific, and targets two, three or more tumor antigens simultaneously. Modular CAR (mCAR) T cells are used as a “living” drug, attached to the adaptor to attack tumor cells. Such a system can be used for treating a variety of diseases, including, without limitation, various cancers (e.g., blood malignancies, solid tumors, etc.).

The CAR-T platform of the present invention includes two parts: (1) a modular CAR (mCAR) that is engineered for reduced toxicity and immunogenicity to human cells; and (2) a precision molecular adaptor (PMA) for targeting the modular CAR-T cells to specific tumor antigen(s). The PMA includes a fixed recognition moiety that is bound by the binding region of mCAR, and a modifiable targeting moiety for binding to two specific tumor antigens.

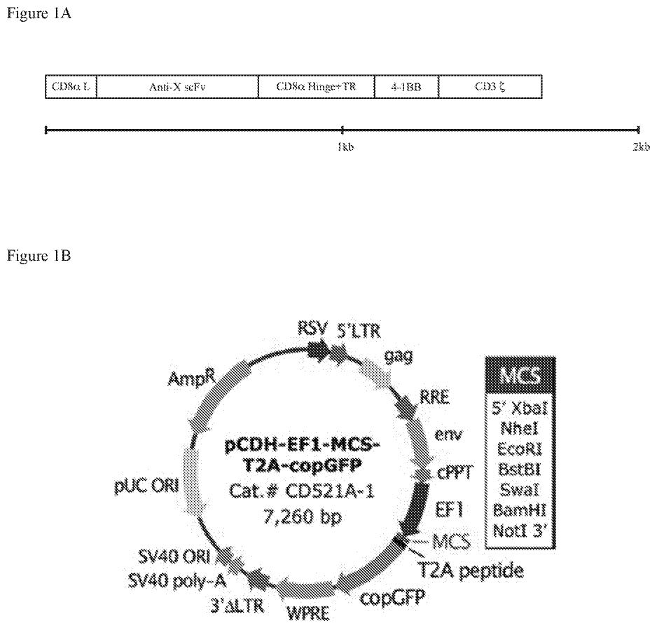

An mCAR includes: a binding region (scFv or other affinity agent) in the extracellular binding domain, a hinge and transmembrane domain, a costimulatory domain, and a T cell signaling domain. The PMA includes: (1) a recognition moiety that includes an indocyanine green (ICG) moiety that is specifically recognized and bound by the binding region of the CAR, (2) one or more targeting moieties, each of which binds specifically to (or is bound specifically by) a respective cell-surface antigens, e.g., antigens such as receptors on the surface of a tumor cell; and optionally (3) a linker that connects and spaces apart the recognition moiety and targeting moiety(-ies). mCAR-T cells can be armed against different tumor targets simply by replacing the PMA (alternatively, the recognition moiety and targeting moiety can be connected directly). An example of an mCAR-T platform of the present invention, including an mCAR DNA construct and a lentiviral vector for introducing the mCAR into T cells, is shown in FIG. 1 and discussed in detail below.

By administration of a PMA along with CAR-expressing T cells (or NK or neutrophil cells) the lymphocyte response can be targeted to only those cells expressing the targeted tumor antigens, thereby reducing off-target toxicity. The recognition moiety (ICG) of the PMA can remain constant; only the targeting moiety(-ies) of the PMA needs to be altered to allow the system to target different cancer cells.

The modular composition of the PMA-mCAR-T platform maintains the high anti-tumor potential of CAR engrafted T cells while introducing real control mechanisms and target flexibility. Advantages of this technology include (1) flexibility to target tumor escape variants, (2) a short development time for adaptors directed against new targets, and (3) rapidly control of the activity of CAR-T cells during therapy. These features allow a more sophisticated application of CAR-T technology and a reduction of adverse events in the clinical setting.

The present invention includes several significant features including:

(1) The use of indocyanine green (ICG) as the binding epitope of PMA for mCAR T cells.

ICG may be used as the binding epitope of the PMA for mCAR T cells, making the system less toxic and less immunogenic.

ICG, a cyanine dye (i.e., a fluorophore), has an excellent safety record and low immunogenicity (Sakka, Curr Opin Crit Care. 13:207-214, 2007; Dzurinko et al., Optometry 75:743-755, 2004). ICG is the only NIRF dye approved by FDA and has been in clinical use since 1959. ICG has been used in clinical diagnostics for over 40 years, e.g., for determining cardiac output, hepatic function, and liver blood flow, and for ophthalmic angiography (Kim et al., Nat Biotechnol 22:93-97, 2004; Soltesz et al., Annals Thoracic Surgery 79:269-277, 2005). The present invention employs a fully human single chain variable fragment (scFv), produced by standard techniques, that binds specifically to ICG as the extracellular binding domain of mCAR to the PMAs.

(2) The use of indocyanine green (ICG) to serve as a “molecular beacon” or “biological GPS” for noninvasive tracking the location of PMA-mCAR-Tcell location with optical imaging in the whole body.

In our PMA-mCAR-T system, the indocyanine green (ICG) in the PMA can serve not only as a recognition epitope for mCAR but also potentially as a molecular beacon (biological GPS) for noninvasively tracking the location and biodistribution of PMA-mCAR-T cells in real time with optical imaging in the whole body.

Neurotoxicity, termed CAR-T-cell-related encephalopathy syndrome (CRES), is the second most-common adverse event in CAR-T cell therapy, and can occur concurrently with or after cytokine-release syndrome (CRS) (Neelapu et al., Nat Rev Clin Oncol. 15:47-62, 2018). Clinical trials of CAR-T therapy have showed that CAR-T cells can cause sever neurotoxicity. Juno Therapeutics abandoned its CAR-T frontrunner JCAR015 after deaths of five patients due to cerebral edema, a neurologic adverse event seen in the pivotal phase II trial(ROCKET) of JCAR015 for adult patients with B-ALL (DeFrancesco, Nat Biotechnol. 35:6-7, 2017). Several study participants also experienced severe neurologic toxicity, akin to what has happened in other trials of CAR-T cells. It has been showed that CAR-T cells can break the blood-brain barrier (BBB), a membranous wall that largely separates the content of blood from the central nervous system to protect the brain, and entered brain to cause severe neurologic toxicity (Dengler, Science Magazine, Nov. 16, 2017, doi:10.1126/science.aar5192). Multiple anti-CD19 CAR-T cell programs have also observed that intravenously infused CAR-T cells can cross the blood-brain barrier to a sufficient degree, irrespective of CNS malignancy status at the time of CAR-T cell therapy (Maude et al. N Engl J Med. 371: 1507-1517, 2014; Davila et al. Sci Transl Med. 6:224ra225, 2014; Mueller et al., Blood 130:2317-2325, 2017; Lee et al., Lancet 385:517-528, 2015; O'Rourke et al., Sci Transl Med 9:399, 2017; Abramson et al., N Engl J Med 377:783-784, 2017).

Intensive monitoring and prompt management of toxicities is essential to minimize the morbidity and mortality associated with this potentially curative therapeutic approach. However, algorithms for accurate and real time tracking the location and distribution of CAR-T cells are lacking. Therefore if we can provide an effective means to monitor and track the location of CAR-T cells directly in vivo after administration during therapy, it will be a powerful tool for the assessment of CAR-T cell-based therapies. By this way, we can intervene early and terminate the CAR-T cells' activity if they appeared in undesired body location and therefore can prevent an adverse event happens.

ICG can be injected into the human blood stream with practically no adverse effects (Alander et al., Int J Biomed Imaging 2012: 940585). ICG becomes fluorescent once excited with specific wavelength light in the near infra-red (NIR) spectrum (approximately 820 nm) (Luo et al., Biomaterials 32:7127-7138) or a laser beam (Daskalaki et al., Surg Innov, doi:10.1177/1553350614524839, 2014, Spinoglio et al., Surg Endosc 27:2156-2162, 2012). The fluorescence can be detected using specific scopes and cameras and then transmitted to a standard monitor allowing identification of anatomical structures where the dye is present (i.e., biliary ducts, vessels, lymph nodes, etc.). Because ICG-based near infra-red fluorescent (NIRF) imaging has advantages such as deep tissue penetration and low autofluorescence, it is applicable to noninvasive in vivo tracking for tumor-specific delivery and biodistribution in living mammals. Among various available imaging techniques, optical imaging (01) has the advantage of being quick, inexpensive, easy to perform, noninvasive, and does not involve ionizing radiation. The first clinical OI scanners have recently entered clinical applications and pilot studies show promising results for investigations of breast tissue in patients (Intes, Acad Radiol 12:934-947, 2005).

ICG has been used in visualizing, tracking and localizing stem cells after transplantation in vivo to help direct and optimize stem cell delivery techniques and confirm successful stem cell deposition into the myocardium (Boddington et al., Cell Transplant 19:55-65, 2010). Direct labelling of human mesenchymal stem cells (hMSC) prior to transplantation provides a means to track cells after administration. The study shows monitoring the real-time fate of in vivo transplanted cells is essential to validate the full potential of stem cells based therapy and potentially for localization of the cell engraftment after transplantation into patients.

In addition, Christensen et al. showed that homing of systemically administered monocytes tagged using indocyanine green (ICG) can be assessed non-invasively using clinically-applicable laser angiography systems to investigate cutaneous inflammatory processes (Christensen et al. 2013, PLOS ONE 8: e81430). Sabapathy et al. also demonstrated that ICG labelled cells can be successfully used for in vivo cell tracking applications in SCID mice injury models (Sabapathy et al., 2015, Stem Cells Int 2015: 606415).

In order to visualize, track and localize stem cells after transplantation in vivo, mCAR-T cells and a PMA are introduced into a patient, as described in detail herein. Binding of the PMA to the CAR-T cells permits NIRF imaging to locate the mCAR-T cells, since the bound PMA includes an ICG moiety.

(3) A Bi-Specific or Tandem PMA

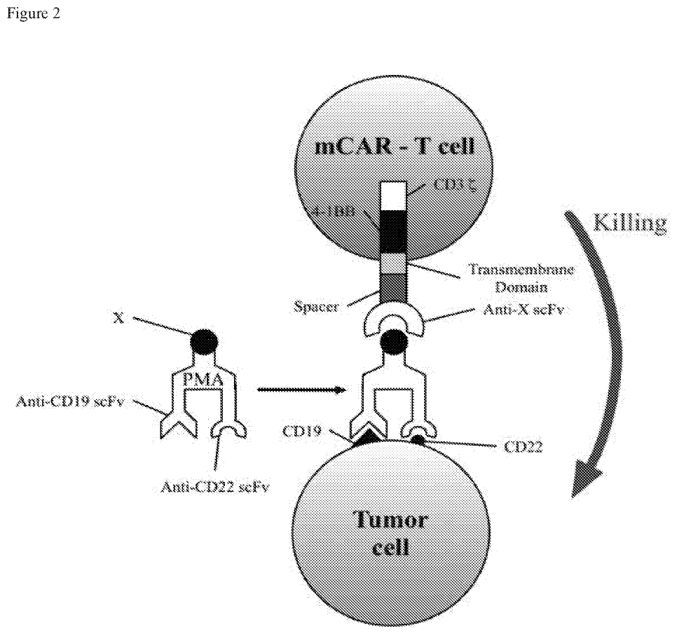

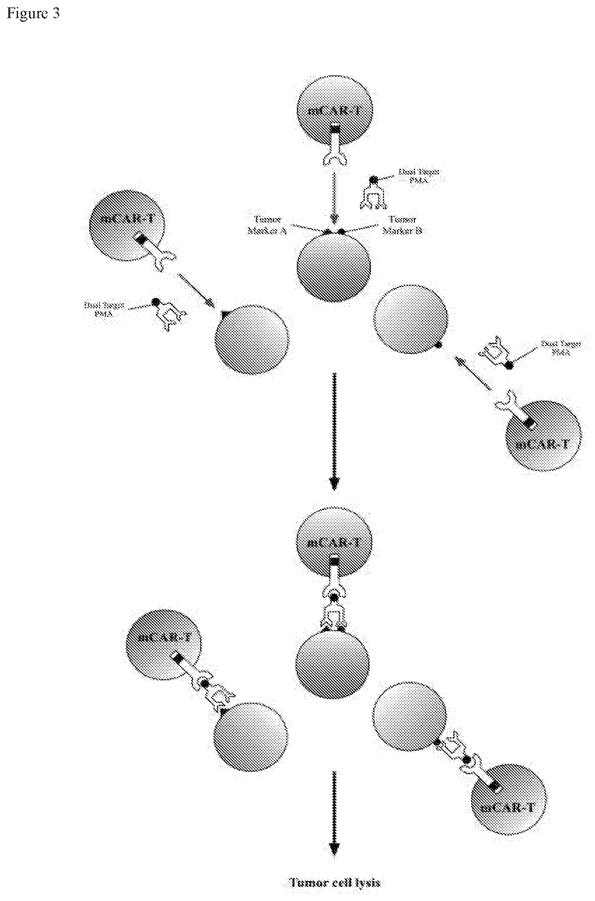

According to one embodiment, the present invention provides a bi-specific PMA incorporating antigen recognition domains (e.g., for CD19 and CD22 for targeting leukemia cells), joined in tandem. Such a PMA-mCAR-T system is shown in FIG. 2. The tandem PMA is used to direct mCAR-T cells to target both antigens simultaneously, to enhance precision and to overcome tumor antigen escape (see FIG. 2 and FIG. 3). The single multi-targeted PMA is interchangeable with a bi-specific PMA, adding a high degree of flexibility to the system.

Dual targeting in conventional CARs has been shown to be more effective at inducing remissions, and could be less susceptible to relapse associated with antigen escape, in glioblastoma (Hegde et al., J Clin Invest 126:3036-3052, 2016) and in B cell malignancies (Zah et al., Cancer Immunol Res 4:498-508, 2016; Ruella et al., J Clin Invest pii: 87366. doi:10.1172/JCI87366, 2016; Schneider et al., J Immunotherapy Cancer 5:42-59, 2017). It is believed that simultaneous immunotherapeutic targeting of multiple antigens may diminish the likelihood of tumor escape through antigen loss (Fry et al., Nature Medicine doi:10.1038/nm.4441, 2017). To effectively prevent antigen escape, the bi-specific CAR not only recognizes two antigens, but also processes both signals in a true Boolean OR-gate fashion—i.e. either antigen input should be sufficient to trigger robust T-cell output (FIG. 3). This particular type of bi-specific CAR is also referred as an “OR-gate CAR” (Zah et al., Cancer Immunol Res 4:498-508, 2016).

The tandem CAR can trigger robust T cell-mediated cytokine production and cytotoxicity when either targeted antigen is present on the target cell (Fry et al., Nature Medicine doi:10.1038/nm.4441, 2017). Simultaneous multi-specific targeting may be a more effective approach to enhance the durability of immunotherapy-induced remission.

For example, in B-cell acute lymphoblastic leukemia (B-ALL), the CD19 antigen is expressed on follicular dendritic cells and B cells. It is present on B cells from the earliest recognizable B-lineage cells during development to B-cell blasts but is lost on maturation to plasma cells. It is expressed on the surface of almost all B cell malignancies but not on hematopoietic stem cells and other tissue cells (Kalos et al., Sci Transl Med 3:95ra73, 2011), so it has been an ideal tumor target. Many centers have designed their own CD19-CAR-T cells, which have proved to be effective and safe in clinical trials (Kochenderfer and Rosenberg, Nat Rev Clin Oncol 10:267-276, 2013; Brentjens et al., Sci Transl Med 5:177ra138, 2013; Lee et al., Lancet 385:517-528, 2015).

The CD22 antigen is another well characterized member of the B-cell antigen family with a tissue distribution that is similar to CD19. CD22 is a 135-kDa sialic acid-binding immunoglobulin-like lectin (SIGLEC) that is expressed exclusively within the B cell lineage. It consists of seven extracellular IgG-like domains and is expressed on the B-cell surface starting at the pre-B cell stage. It persists on mature B cells and is lost on plasma cells (Nitschke, Immunol Rev 230:128-143, 2009). CD22 is one of the most commonly displayed antigens in hematologic B cell malignancies, including human B-cell lymphomas and leukemias (Clark, J Immunol 150:4715-4718, 1993; Robbins et al., Blood 82:1277-1287, 1993). CD22 is displayed in 96% to 100% of cases of pediatric acute lymphoblastic leukemia (ALL) (Gudowius et al., Klin Padiatr 218:327-333, 2006; Olejniczak, Immunol Invest 35:93-114, 2006), more than 90% of cases of chronic lymphocytic leukemia (CLL) (Rawstron, Leukemia 20:2102-2110, 2006), 60% to 70% of B-cell lymphomas (Clark, J Immunol 150:4715-4718, 1993), and 100% of hairy cell leukemia (HCL) (Clark, J Immunol 150:4715-4718, 1993). CD22-CAR T cells have a similar safety profile to that of CD19-CAR T cells and mediate similarly potent anti-leukemic effects in both immunotherapy-naive patients and patients with CD19dim or CD19-relapse following CD19-directed immunotherapy (Fry et al., Nature Medicine doi:10.1038/nm.4441, 2017; Haso et al., Blood 121:1165-1174, 2013).

An approach that targets both CD19 and CD22 is expected to have improved effectiveness in killing B-ALL tumor cells.

Precision Molecular Adaptors (PMA)

The CAR system of the present invention utilizes PMAs (also referred to as “adapters” or “switches”), small conjugate molecules that serve as the bridge between cytotoxic lymphocytes and targeted cancer cells. PMAs include an ICG (or other recognition moiety) at one end and a targeting moiety on the other, optionally connected by a linker or bridge domain. The recognition moiety is a molecule, e.g., ICG, that is recognized and specifically bound by a CAR. Exemplary targeted moieties include ICG and ICG derivatives, including without limitation cypate and cypate derivatives, for example cytate (cypateoctreote peptide analog conjugate) and cybesin (cypate-bombesin peptide analog conjugate), and methylene blue (methylthioninium chloride).

As used herein, the terms “indocyanine green” and “ICG” are used generically to refer to ICG, ICG derivatives, and methylene blue.

The PMA also includes a targeting moiety, an affinity agent (as defined below) that binds to one or more cell surface antigens such as receptor ligands of the targeted cell, e.g., a tumor cell. Although any targeting moiety that binds to a cell associated with a disease may be utilized, in various embodiments the antibody that specifically binds to a tumor-associated antigen (TAA). Examples of tumor-associated antigens include, without limitation, alpha-fetoprotein (AFP), α-actinin-4, A3, antigen specific for A33 antibody, ART-4, B7, Ba 733, BAGE, BrE3-antigen, members of the carbohydrate antigen family, members of the carbonic anhydrase family, CA125, CAMEL, CAP-1, CASP-8/m, CCL19, CCL21, CD1, CD1a, CD2, CD3, CD4, CD5, CD8, CD10, CD11A, CD14, CD15, CD16, CD18, CD19, CD20, CD21, CD22, CD23, CD25, CD29, CD30, CD32b, CD33, CD37, CD38, CD40, CD40L, CD44, CD45, CD46, CD52, CD54, CD55, CD59, CD64, CD66a-e, CD67, CD70, CD70L, CD74, CD79a, CD79b, CD80, CD83, CD95, CD99, CD 123, CD126, CD132, CD133, CD138, CD147, CD154, CD274, CDC27, CDK-4/m, CDKN2A, CTLA4, CXCR4, CXCR7, CXCL12, HIF-1α, colon-specific antigen-p (CSAp), CEA (CEACAM-5), CEACAM-6, c-Met, DAM, diasialogangliosides, embryonic antigens, members of the epidermal growth factor receptor family and mutants thereof (e.g., EGFR and EGFRvIII), members of the ephithelia glycoprotein family, EGP-1 (TROP-2), EGP-2, ELF2-M, Ep-CAM, members of the ephrin receptor family, erb-1, erb-2, fibroblast growth factor (FGF), Flt-1, Flt-3, folate binding protein (FBP), α-folate receptor, follicle stimulating hormone receptor, G250 antigen, GAGE, gp100, members of the Rho family of GTPases, GRO-β, HLA-DR, HM1.24, human chorionic gonadotropin (HCG) and its subunits, HER2/neu, members of the high mobility group proteins and mutants thereof, HMGB-1, human high molecular weight-melanoma-associated antigen, hypoxia inducible factor (HIF-1), HSP70-2M, HST-2, Ia, IGF-1R, IFN-γ, IFN-α, IFN-β, IFN-k, IL-4R, IL-6R, IL-13R, IL-15R, IL-17R, IL-18R, IL-2, IL-6, IL-8, IL-12, IL-15, IL-17, IL-18, IL-23, IL-25, insulin-like growth factor-1 (IGF-1), KC4-antigen, KS-1-antigen, KS1-4, Le-Y, LDR/FUT, ligands of the NKG2D receptor, macrophage migration inhibitory factor (MIF), MAGE, MAGE-3, MART-1, MART-2, NY-ESO-1, TRAG-3, mCRP, MCP-1, MIP-1A, MIP-1B, MIF, members of the mucin protein family (e.g., MUC1, MUC2, MUC3, MUC4, MUC5ac, MUC13, MUC16), MUM-1/2, MUM-3, NCA66, NCA95, NCA90, pancreatic cancer mucin, PD1 receptor, placental growth factor, p53, PLAGL2, prostatic acid phosphatase, prostrate specific antigens (e.g., PSA, PSCA or PSMA), PRAME, PlGF, ILGF, ILGF-R, IL-6, IL-25, RS5, RANTES, T101, SAGE, S100, survivin, survivin-2B, TAC, TAG-72, tenascin, TIM-3, TRAIL receptors, TNF-α, Tn antigen, Thomson-Friedenreich antigens, tumor necrosis antigens, members of the vascular endothelial growth factor receptor (VEGFR) family, ED-B fibronectin, WT-1, 17-1A-antigen, complement factors C3, C3a, C3b, C5a, C5, an angiogenesis marker, bcl-2, bcl-6, Kras, an oncogene marker or an oncogene product (see, e.g., Sensi et al., Clin Cancer Res 2006, 12:5023-32; Parmiani et al., J Immunol 2007, 178:1975-79; Novellino et al. Cancer Immunol Immunother 2005, 54:187-207), and wherein the binding moiety of target modules is composed of the alpha and beta or the gamma and delta chains of a T cell receptor or fragments thereof, including auto-reactive T cell receptor-derived receptors, wherein such T cell receptor-derived binding moieties recognize and bind to peptides presented by human leukocyte antigen class I and II protein complexes.

Exemplary antibodies against TAAs include, but are not limited to, hA19 (anti-CD19, U.S. Pat. No. 7,109,304), hR1 (anti-IGF-1R, U.S. patent application Ser. No. 13/688,812, filed Mar. 12, 2010), hPAM4 (anti-MUC5ac, U.S. Pat. No. 7,282,567), hA20 (anti-CD20, U.S. Pat. No. 7,151,164), hIMMU31 (anti-AFP, U.S. Pat. No. 7,300,655), hLL1 (anti-CD74, U.S. Pat. No. 7,312,318), hLL2 (anti-CD22, U.S. Pat. No. 5,789,554), hMu-9 (anti-CSAp, U.S. Pat. No. 7,387,773), hL243 (anti-HLA-DR, U.S. Pat. No. 7,612,180), hMN-14 (anti-CEACAM-5, U.S. Pat. No. 6,676,924), hMN-15 (anti-CEACAM-6, U.S. Pat. No. 8,287,865), hRS7 (anti-EGP-1, U.S. Pat. No. 7,238,785), hMN-3 (anti-CEACAM-6, U.S. Pat. No. 7,541,440), hRFB4 (anti-CD22, U.S. Pat. No. 9,139,649), Ab124 and Ab125 (anti-CXCR4, U.S. Pat. No. 7,138,496). Fragments of such antibodies are useful as affinity agents in the practice of the present invention.

The recognition moiety and the targeting moiety can be directly conjugated by standard techniques. However, the use of a linking domain, or linker, to connect the two molecules can be helpful as it can provide flexibility and stability to the PMA. Examples of suitable linking domains include: polyethylene glycol (PEG); polyproline; hydrophilic amino acids; sugars; unnatural peptideoglycans; polyvinylpyrrolidone; and pluronics, e.g., pluronic F-127. Linkers lengths that are suitable include, but are not limited to, linkers having 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40, or more atoms.

While the affinity at which the targeting moiety binds to its target can vary, and in some cases low affinity binding may be preferable (such as about 1 μM), the binding affinity of the targeting moiety to its targetwill generally be at least about 100 μM, 1 nM, 10 nM, or 100 nM, preferably at least about 1 pM or 10 pM, even more preferably at least about 100 pM.

Prior to being administered to a subject, the PMAs are prepared in a pharmaceutically acceptable formulation. Such formulations may contain a pharmaceutically acceptable carrier or diluent.

Chimeric Antigen Receptors (CARs)

The CAR system of the present invention also utilizes cytotoxic lymphocytes engineered to express a chimeric antigen receptor (CAR) that recognizes and binds the recognition moiety of a PMA. The CARs used in the CAR system comprise three domains, e.g., in the form of a fusion protein. The first domain is the binding region which, as the name suggests, recognizes and binds the recognition moiety of the PMA. The second domain is the co-stimulation domain, which enhances the proliferation and survival of the lymphocytes. The third domain is the activation signaling domain, which is a cytotoxic lymphocyte activation signal.

The binding region of the CAR is an affinity agent, e.g., a single chain fragment variable (scFv) regions of an antibody that binds the recognition moiety of a PMA. Preferably, the scFv regions bind the recognition moiety with specificity. The identity of the affinity agent used in the production of the binding region is limited only in that it binds specifically to the recognition moiety of the PMA. The scFv regions can be prepared from (i) antibodies known in the art that bind a recognition moiety, (ii) antibodies newly prepared using a selected recognition moiety as a hapten, and (iii) sequence variants derived from the scFv regions of such antibodies, e.g., scFv regions having at least about 80% sequence identity to the amino acid sequence of the scFv region from which they are derived.

The co-stimulation domain serves to enhance the proliferation and survival of the cytotoxic lymphocytes upon binding of the CAR to a targeted moiety. The identity of the co-stimulation domain is limited only in that it has the ability to enhance cellular proliferation and survival activation upon binding of the targeted moiety by the CAR. Suitable co-stimulation domains include, without limitation: CD28 (Alvarez-Vallina et al., Eur J Immunol 26:2304-2309, 1996); CD137 (4-1BB), a member of the tumor necrosis factor (TNF) receptor family (Imai et al., Leukemia 18:676-684, 2004); CD134 (OX40), a member of the TNFR-superfamily of receptors (Latza et al., Eur J Immunol 24:677, 1994); CD278 (ICOS), a CD28-superfamily co-stimulatory molecule expressed on activated T cells (Hutloff et al., Nature 397:263, 1999); other co-stimulation domains include without limitation: CD2, CD7, CD27, CD30, CD40, DAP10, DAP12, programmed cell death-1 (PD-1), cytotoxic T-lymphocyte antigen 4 (CTLA-4), CD276 (B7-H3), lymphocyte function-associated antigen-1 (LFA-1), LIGHT, CD159c (NKG2C), and a ligand that specifically binds with CD83. Sequence variants of these noted co-stimulation domains can be used without adversely impacting the invention, where the variants have the same or similar activity as the domain on which they are modeled. Such variants will commonly have at least about 80% sequence identity to the amino acid sequence of the domain from which they are derived.

In some embodiments of the invention, the CAR constructs comprise two co-stimulation domains. While the particular combinations include all possible variations of the four noted domains, specific examples include: CD28+CD137 (4-1BB) and CD28+CD134 (OX40).

The activation signaling domain serves to activate cytotoxic lymphocytes upon binding of the CAR to a targeted moiety. The identity of the activation signaling domain is limited only in that it has the ability to induce activation of the selected cytotoxic lymphocyte upon binding of the recognition moiety of the PMA by the CAR. Suitable activation signaling domains include the T cell CD3 chain and Fc receptor γ. Sequence variants of these noted activation signaling domains can be used without adversely impacting the invention, where the variants have the same or similar activity as the domain on which they are modeled. Such variants will commonly have at least about 80% sequence identity to the amino acid sequence of the domain from which they are derived.

Constructs encoding the CARs of the invention are prepared through genetic engineering. As an example, a plasmid or viral expression vector can be prepared that encodes a fusion protein comprising a binding region, one or more co-stimulation domains, and an activation signaling domain, in frame and linked in a 5′ to 3′ direction. However, the CARs of the present invention are not limited in this arrangement and other arrangements are acceptable and include: (i) a binding region, an activation signaling domain, and one or more co-stimulation domains, and (ii) a binding region, a co-stimulation domain, and an activation signaling domain, linked in a 5′ to 3′ direction. Because the binding region must be free to bind the targeted moiety, the placement of the binding region in the fusion protein will generally be such that display of the region on the exterior of the cell is achieved. In the same manner, because the co-stimulation and activation signaling domains serve to induce activity and proliferation of the cytotoxic lymphocytes, the constructs will generally encode a fusion protein that displays these two domains in the interior of the cell.

The CARs may include additional elements, such a signal peptide to ensure proper export of the fusion protein to the cells surface, a transmembrane domain to ensure the fusion protein is maintained as an integral membrane protein, and a hinge domain that imparts flexibility to the recognition region and allows strong binding to the targeted moiety.

An exemplary CAR construct is shown in FIG. 1A. The construct includes: a signal peptide sequence (CD8a leader sequence, CD8a L), anti-ICG scFV, CD8a hinge and transmembrane domain (CD8a Hinge+TR), 4-1BB and CD3ζ.

In addition to the use of plasmid and viral vectors, cytotoxic lymphocytes can be engineered to express CARs of the invention through retrovirus, lentivirus (viral mediated CAR gene delivery system), sleeping beauty, and piggyback (transposon/transposase systems that include a non-viral mediated CAR gene delivery system).

While the affinity at which the CARs, expressed by the cytotoxic lymphocytes, bind to the recognition moiety of the PMA can vary, and in some cases low affinity binding may be preferable (such as about 50 nM), the binding affinity of the CARs to the targeted ligand will generally be at least about 100 nM, 1 pM, or 10 pM, preferably at least about 100 pM, 1 fM or 10 fM, even more preferably at least about 100 fM.

Although the majority of CAR, CAR-T and CAR-NK constructs have been based on the scFv antibody fragment for disease cell targeting, use of other antibody fragments has also been disclosed for this purpose. For example, Nolan et al. (Clin Cancer Res 5:3928-3941, 1999) disclosed use of anti-CEA Fab antibody fragments to make chimeric immunoglobulin-T cell receptors. Fab fragments were found to be as effective as scFv fragments for expression and antigen binding. Fab fragments may be advantageous over scFv fragments in terms of stability of antigen-binding affinity.

The CAR sequences will be incorporated in an expression vector. Various expression vectors are known in the art and any such vector may be utilized. In one embodiment, the vector is a retroviral or lentiviral vector. Techniques for genetic manipulation of NK cells for cancer immunotherapy have been discussed by Carlsten & Childs (2015, Front Immunol 6:266). Viral vectors used for NK cell infection have primarily included retroviral and lentiviral vectors (Carlsten & Childs, 2015). However, decreased viability of primary NK cells undergoing retroviral transduction may limit this approach (Carlsten & Childs, 2015). Lentiviral transduction has been somewhat more effective, with efficiencies of 15 to 40% (Carlsten & Childs, 2015). Transfection by electroporation or lipofection is reported to result in lower induction of apoptosis than viral transduction, with more rapid but transient expression of the transgene(s) (Carlsten & Childs, 2015). Strategies used to increase efficacy have included transduction with IL-2 or IL-15 (promoting clone persistence and expansion), CCR7 and CXCR3 to improve migration, and various genes such as CARs, CD17, IL-2, IL-15, NKG2A and double negative TGF-β II receptor to increase cytotoxicity. The skilled artisan will realize that these and other effectors known to be of use for CAR, CAR-T and CAR-NK constructs may be utilized in the instant methods and compositions.

CAR-Expressing Effector Cells

The effector cells used in the CAR system of the present invention including any suitable cells known in the art, e.g., are lymphoid lineage cells, including without limitation T cells, natural killer (NK) cells, cytotoxic T lymphocytes (CTLs), and regulatory T cells; myeloid lineage cells, including without limitation neutrophils and macrophages; and other suitable cells, including without limitation rδ T cells, Natural Killer T cells (NKT cells), and lymphokine-activated killer (LAK) cells. Upon activation, the effector cells triggers the destruction of target tumor cells. For example, cytotoxic T cells trigger the destruction of target tumor cells by either or both of the following means. First, upon activation T cells release cytotoxins such as perforin, granzymes, and granulysin. Perforin and granulysin create pores in the target cell, and granzymes enter the cell and trigger a caspase cascade in the cytoplasm that induces apoptosis (programmed cell death) of the cell. Second, apoptosis can be induced via Fas-Fas ligand interaction between the T cells and target tumor cells.

Herein, the term “CAR-T” refers not only to T cells but also to other cell types that are engineered to express a CAR construct.

The cytotoxic lymphocytes will preferably be autologous cells, although heterologous cells can also be used, such as when the subject being treated using the CAR system of the invention has received high-dose chemotherapy or radiation treatment to destroy the subject's immune system. Under such circumstances, allogenic cells can be used.

The cytotoxic lymphocytes can be isolated from peripheral blood using techniques well known in the art, include Ficoll density gradient centrifugation followed by negative selection to remove undesired cells.

Cytotoxic lymphocytes can be engineered to express CAR constructs by transfecting a population of lymphocytes with an expression vector encoding the CAR construct. Appropriates means for preparing a transduced population of lymphocytes expressing a selected CAR construct will be well known to the skilled artisan, and includes retrovirus, lentivirus (viral mediated CAR gene delivery system), sleeping beauty, and piggyback (transposon/transposase systems that include a non-viral mediated CAR gene delivery system), to name a few examples.

Transduced cytotoxic lymphocytes are grown in conditions that are suitable for a population of cells that will be introduced into a subject such as a human. Specific considerations include the use of culture media that lacks any animal products, such as bovine serum. Other considerations include sterilized-condition to avoid contamination of bacteria, fungi and mycoplasma.

Prior to being administered to a subject, the cells are pelleted, washed, and resuspended in a pharmaceutically acceptable carrier or diluent. Exemplary formulations comprising CAR-expressing cytotoxic lymphocytes include formulations comprising the cells in sterile 290 mOsm saline, infusible cryomedia (containing Plasma-Lyte A, dextrose, sodium chloride injection, human serum albumin and DMSO), 0.9% NaCl with 2% human serum albumin or any other sterile 290 mOsm infusible materials.

“Universal” or Allogeneic T Cells

New T cell sources can reduce the need for autologous cell manufacturing and enable cell transfer across histocompatibility barriers. CAR-modified allogeneic cells have the potential to act as universal effector cells, which can be administered to any patient regardless of major histocompatibility complex (MEW) type. Such universal effector cells could be used as an “off-the-shelf” cell-mediated treatment for cancer.

Allogeneic T Cells

While T cells can be easily harvested from donors, their use is compromised by their high alloreactive potential. Owing to their ontogeny, T-cell receptors (TCRs) are naturally prone to react against non-autologous tissues, recognizing either allogeneic human leukocyte antigen (HLA) molecules or other polymorphic gene products, referred to as minor antigens (Afzali et al., Tissue Antigens 69, 545-556, 2007). This propensity underlies the high risk of graft rejection in transplant recipients and of graft-versus-host disease (GVHD) in recipients of donor-derived T cells. Thus, bulk unselected donor T cells are prone to cause normal tissue destruction and may be lethal on occasion. To provide an acceptable risk-benefit ratio, allogeneic T cells must be devoid of alloreactive potential. Two strategies designed to overcome the risk of graft-versus-host (GVH) reactions have been proposed, based on the selection of virus-specific TCRs devoid of GVH reactivity or the ablation of TCR expression.

1) Virus-Specific T Cells

Recent studies have suggested that donor-derived virus-specific T cells can be administered to multiple recipients with limited risk of GVHD (Doubrovina et al., Blood 119: 2644-2656, 2012; Haque et al., Blood 110: 1123-1131, 2007). Virus-specific T cells may thus serve as cellular vehicles for TCR or CAR therapy. A first trial testing this approach showed that T cells expanded in vivo in response to viral reactivation although anti-tumor activity was modest (Cruz et al., Blood 122:2965-2973, 2013). While the relatively limited expansion potential of virus-specific T cells and the sometimes unpredictable cross-reactivity of TCR-mediated antigen recognition are valid concerns, this approach to treat viral infections represents a first step toward multi-recipient T-cell product manufacturing (Wang and Rivière, Cancer Gene Ther 22: 85-94, 2015).

2) Genome Editing of Allogeneic T Cells

If the endogenous TCR of T cells from donor peripheral blood cannot be tamed, one may abrogate its expression, making the engineered TCR or CAR the sole driver of T cell activation and clonal expansion. Gene-disrupted allogeneic T cells could provide an alternative as a universal donor to autologous T cells. Four technologies based on the use of targeted nucleases, including meganucleases, zinc-finger nucleases (ZFNs), transcription activator-like effector nucleases (TALEN), and CRISPR/Cas9, enable gene disruption in human cells (Kim and Kim, Nat Rev Genet 15:321-334, 2014; Sander and Joung, Nat Biotechnol 32:347-355, 2014). The ablation of endogenous TCR expression has been achieved using targeted ZFNs or TALENs that disrupt the constant regions of TCRA and TCRB genes (Berdien et al., Gene Ther 21:539-548, 2014; Provasi et al., Nat Med 18:807-815, 2012; Torikai et al., Blood 119: 5697-5705, 2012). Multiplex ablation of endogenous TCR, HLA class I molecule (beta-2 microglobulin (B2M)) and PD1 simultaneously in donor derived allogeneic T cells using CRISPR/Cas9 has been reported recently to generate universal T cells deficient of TCR, HLA class I molecule and PD1 (Ren et al., Clin Cancer Res, November 4. pii: clincanres.1300.2016, 2016).

Lymphoid Progenitor

While T cells can cause graft-versus-host disease (GVHD), their precursors do not, as they undergo positive and negative selection in the recipient's thymus. Taking advantage of this requires the ability to expand T cell precursors in culture, which is now possible due to advances in understanding T cell development (Awong et al., Semin Immunol 19:341-349, 2007; Rothenberg, J Immunol 186:6649-6655, 2011; Shah and Zaiga-Pflücker, J Immunol 192:4017-4023, 2014). T cell precursors lack the ability to initiate GVH reactions because they complete their differentiation in the recipient's thymus wherein they become restricted to host MHC and yield T lymphocytes that are host tolerant (Zakrzewski et al., Nat Med 12:1039-1047, 2006). When transduced with a CAR, allogeneic lymphoid progenitors yield tumor-targeted T cells without causing GVHD (Zakrzewski et al., Nat Biotechnol 26:453-461, 2008). The main advantage of using T cell precursors for immunotherapy is that this approach does not require strict histocompatibility between donors and recipients. In mice, this therapy works with unrelated fully mismatched cells just as well as with autologous cells. T cell precursor immunotherapy may therefore allow for a true “off-the-shelf” therapy, if lymphoid progenitor cell manufacturing can be scaled up (Themeli et al., Cell Stem Cell, 16:357-366, 2015).

Hematopoietic Stem Cells (HSCs)

As with gene modification of HSCs with TCRs, modification of HSCs with genes encoding CARs brings the prospect of long-term production of immune effector cells targeted to tumor-associated antigens. One major potential advantage with CARs is that their expression will not be limited to T cells, as is the case with TCR genes introduced into HSCs, since the surface display of CARs does not require CD3 co-expression. Thus, CARs may be expressed on multiple hematopoietic lineages (including without limitation lymphoid lineage cells such as T or NK cells, and myeloid lineage cells such as neutrophils), amplifying the potential graft-versus-cancer activity (Kohn et al., Biol Blood Marrow Transplant 19:S64-S69, 2013; Hege et al., J Exp Med 184:2261-2269, 1996; Roberts et al., J Immunol 161:375-384, 1998; Badowski et al., J Exp Ther Oncol 8:53-63, 2009; Doering et al., Adv Drug Delivery Rev., 62:1204-1212, 2010). The multi-lineage expression of CARs, associated with potent engineered antigen-specific cytotoxicity, makes the CAR modification of HSC a very promising cancer immunotherapy approach to be explored.

Pluripotent Stem Cells

Another approach is to generate ideal T cells artificially rather than modify those naturally formed. Pluripotent stem cells can give rise to a variety of somatic cells (Inoue et al., EMBO J 33:409-417, 2014; Murry and Keller, Cell 132:661-680, 2008; Takahashi et al., Cell 131:861-872, 2007) and thus have in principle the potential to serve as an endless supply of therapeutic T lymphocytes. A few reports support the feasibility of generating T lymphocytes from human ESCs and iPSCs in vitro (Kennedy et al., Cell Rep. 2:1722-1735, 2012; Nishimura et al., Cell Stem Cell 12:114-126, 2013; Themeli et al., Nat Biotechnol 31:928-933, 2013; Timmermans et al., J Immunol 182:6879-6888, 2009; Vizcardo et al., Cell Stem Cell 12:31-36, 2013).

Antibodies

General Antibody Techniques

Techniques for preparing monoclonal antibodies against virtually any target antigen are well known in the art. See, for example, Kohler and Milstein, Nature 256: 495 (1975), and Coligan et al. (eds.), Current Protocols Immunology, Vol. 1, pages 2.5.1-2.6.7 (John Wiley & Sons 1991). Briefly, monoclonal antibodies can be obtained by injecting mice with a composition comprising an antigen, removing the spleen to obtain B-lymphocytes, fusing the B-lymphocytes with myeloma cells to produce hybridomas, cloning the hybridomas, selecting positive clones which produce antibodies to the antigen, culturing the clones that produce antibodies to the antigen, and isolating the antibodies from the hybridoma cultures. MAbs can be isolated and purified from hybridoma cultures by a variety of well-established techniques. Such isolation techniques include affinity chromatography with Protein-A Sepharose, size-exclusion chromatography, and ion-exchange chromatography. See, for example, Coligan at pages 2.7.1-2.7.12 and pages 2.9.1-2.9.3. Also, see Baines et al., “Purification of Immunoglobulin G (IgG),” in METHODS IN MOLECULAR BIOLOGY, VOL. 10, pages 79-104 (The Humana Press, Inc. 1992).

After the initial raising of antibodies to an immunogen, the antibodies can be sequenced and subsequently prepared by recombinant techniques. Humanization and chimerization of murine antibodies and antibody fragments are well known to those skilled in the art.

Chimeric Antibodies

A chimeric antibody is a recombinant protein in which the variable regions of a human antibody have been replaced by the variable regions of, for example, a mouse antibody, including the complementarity-determining regions (CDRs) of the mouse antibody. Chimeric antibodies exhibit decreased immunogenicity and increased stability when administered to a subject. General techniques for cloning murine immunoglobulin variable domains are disclosed, for example, in Orlandi et al., Proc. Nat'l Acad. Sci. USA 86: 3833 (1989). Techniques for constructing chimeric antibodies are well known.

Humanized Antibodies

Techniques for producing humanized MAbs are well known (see, e.g., Jones et al., Nature 321: 522 (1986), Riechmann et al., Nature 332: 323 (1988), Verhoeyen et al., Science 239: 1534 (1988), Carter et al., Proc. Nat Acad. Sci. USA 89: 4285 (1992), Sandhu, Crit. Rev. Biotech. 12: 437 (1992), and Singer et al., J. Immun. 150: 2844 (1993)). A chimeric or murine monoclonal antibody may be humanized by transferring the mouse CDRs from the heavy and light variable chains of the mouse immunoglobulin into the corresponding variable domains of a human antibody. The mouse framework regions (FR) in the chimeric monoclonal antibody are also replaced with human FR sequences. As simply transferring mouse CDRs into human FRs often results in a reduction or even loss of antibody affinity, additional modification might be required in order to restore the original affinity of the murine antibody. This can be accomplished by the replacement of one or more human residues in the FR regions with their murine counterparts to obtain an antibody that possesses good binding affinity to its epitope. See, for example, Tempest et al., Biotechnology 9:266 (1991) and Verhoeyen et al., Science 239: 1534 (1988). Generally, those human FR amino acid residues that differ from their murine counterparts and are located close to or touching one or more CDR amino acid residues would be candidates for substitution.

Human Antibodies

Methods for producing fully human antibodies using either combinatorial approaches or transgenic animals transformed with human immunoglobulin loci are known in the art (e.g., Mancini et al., 2004, New Microbiol. 27:315-28; Conrad and Scheller, 2005, Comb. Chem. High Throughput Screen. 8:117-26; Brekke and Loset, 2003, Curr. Opin. Pharmacol. 3:544-50). A fully human antibody also can be constructed by genetic or chromosomal transfection methods, as well as phage display technology, all of which are known in the art. See for example, McCafferty et al., Nature 348:552-553 (1990). Such fully human antibodies are expected to exhibit even fewer side effects than chimeric or humanized antibodies and to function in vivo as essentially endogenous human antibodies. In certain embodiments, the claimed methods and procedures may utilize human antibodies produced by such techniques.

In one alternative, the phage display technique may be used to generate human antibodies (e.g., Dantas-Barbosa et al., 2005, Genet. Mol. Res. 4:126-40). Human antibodies may be generated from normal humans or from humans that exhibit a particular disease state, such as cancer (Dantas-Barbosa et al., 2005). The advantage to constructing human antibodies from a diseased individual is that the circulating antibody repertoire may be biased towards antibodies against disease-associated antigens.

In one non-limiting example of this methodology, Dantas-Barbosa et al. (2005) constructed a phage display library of human Fab antibody fragments from osteosarcoma patients. Generally, total RNA was obtained from circulating blood lymphocytes (Id.). Such libraries may be screened by standard phage display methods, as known in the art (see, e.g., Pasqualini and Ruoslahti, 1996, Nature 380:364-366; Pasqualini, Quart J Nucl Med 43:159-162, 1999).

Phage display can be performed in a variety of formats, for their review, see e.g. Johnson and Chiswell, Current Opinion in Structural Biology 3:5564-571 (1993). Human antibodies may also be generated by in vitro activated B cells. See U.S. Pat. Nos. 5,567,610 and 5,229,275. Any known method for making and screening human antibodies or antibody fragments may be utilized.

In another alternative, transgenic animals that have been genetically engineered to produce human antibodies may be used to generate antibodies against essentially any immunogenic target, using standard immunization protocols. Methods for obtaining human antibodies from transgenic mice are disclosed by Green et al., Nature Genet. 7:13 (1994), Lonberg et al., Nature 368:856 (1994), and Taylor et al., Int. Immun. 6:579 (1994). A non-limiting example of such a system is the Xenomouse® (e.g., Green et al., 1999, J. Immunol. Methods 231:11-23) from Abgenix (Fremont, Calif.). In these and similar animals, the mouse antibody genes have been inactivated and replaced by functional human antibody genes, while the remainder of the mouse immune system remains intact.

The Xenomouse was transformed with germline-configured YACs (yeast artificial chromosomes) that contained portions of the human IgH and Igkappa loci, including the majority of the variable region sequences, along accessory genes and regulatory sequences. The human variable region repertoire may be used to generate antibody producing B cells, which may be processed into hybridomas by known techniques. A Xenomouse immunized with a target antigen will produce human antibodies by the normal immune response, which may be harvested and/or produced by standard techniques discussed above. A variety of strains of Xenomouse are available, each of which is capable of producing a different class of antibody. Transgenically produced human antibodies have been shown to have therapeutic potential, while retaining the pharmacokinetic properties of normal human antibodies (Green et al., 1999). The skilled artisan will realize that the claimed compositions and methods are not limited to use of the Xenomouse system but may utilize any transgenic animal that has been genetically engineered to produce human antibodies.

Antibody Cloning and Production

Various techniques, such as production of chimeric or humanized antibodies, may involve procedures of antibody cloning and construction. The antigen-binding Vκ (variable light chain) and VH (variable heavy chain) sequences for an antibody of interest may be obtained by a variety of molecular cloning procedures, such as RT-PCR, 5′-RACE, and cDNA library screening. The V genes of an antibody from a cell that expresses a murine antibody can be cloned by PCR amplification and sequenced. To confirm their authenticity, the cloned VL and VH genes can be expressed in cell culture as a chimeric Ab as described by Orlandi et al., (Proc. Natl. Acad Sci. USA, 86: 3833 (1989)). Based on the V gene sequences, a humanized antibody can then be designed and constructed, e.g., as described by Leung et al. (Mol. Immunol., 32: 1413, 1995).

cDNA can be prepared from any known hybridoma line or transfected cell line producing a murine antibody by general molecular cloning techniques (Sambrook et al., Molecular Cloning, A laboratory manual, 2nd Ed (1989)). The Vκ sequence for the antibody may be amplified using the primers VK1BACK and VK1FOR (Orlandi et al., 1989) or the extended primer set described by Leung et al. (BioTechniques, 15: 286 (1993)). The VH sequences can be amplified using the primer pair VH1BACK/VH1FOR (Orlandi et al., 1989) or the primers annealing to the constant region of murine IgG described by Leung et al. (Hybridoma, 13:469 (1994)). Humanized V genes can be constructed by a combination of long oligonucleotide template syntheses and PCR amplification as described by Leung et al. (Mol. Immunol., 32: 1413 (1995)).

PCR products for Vκ can be subcloned into a staging vector, such as a pBR327-based staging vector, VKpBR, that contains an Ig promoter, a signal peptide sequence and convenient restriction sites. PCR products for VH can be subcloned into a similar staging vector, such as the pBluescript-based VHpBS. Expression cassettes containing the Vκ and VH sequences together with the promoter and signal peptide sequences can be excised from VKpBR and VHpBS and ligated into appropriate expression vectors, such as pKh and pG1g, respectively (Leung et al., Hybridoma, 13:469 (1994)). The expression vectors can be co-transfected into an appropriate cell and supernatant fluids monitored for production of a chimeric, humanized or human antibody. Alternatively, the Vκ and VH expression cassettes can be excised and subcloned into a single expression vector, such as pdHL2 (Gillies et al., J Immunol Methods 125:191, 1989; Losman et al., Cancer 80:2660, 1997).

In an alternative embodiment, expression vectors may be transfected into host cells that have been pre-adapted for transfection, growth and expression in serum-free medium. Exemplary cell lines that may be used include the Sp/EEE, Sp/ESF and Sp/ESF-X cell lines (see, e.g., U.S. Pat. Nos. 7,531,327; 7,537,930; and 7,608,425).

Antibody Fragments

Antibody fragments that recognize specific epitopes can be generated by known techniques. Antibody fragments are antigen binding portions of an antibody, such as F(ab′)2, Fab′, F(ab)2, Fab, Fv, scFv and the like. F(ab′)2 fragments can be produced by pepsin digestion of the antibody molecule and Fab′ fragments can be generated by reducing disulfide bridges of the F(ab′)2 fragments. Alternatively, Fab′ expression libraries can be constructed (Huse et al., 1989, Science, 246:1274-1281) to allow rapid and easy identification of monoclonal Fab′ fragments with the desired specificity. F(ab)2 fragments may be generated by papain digestion of an antibody.

A single chain Fv molecule (scFv) comprises a VL domain and a VH domain. The VL and VH domains associate to form a target binding site. These two domains are further covalently linked by a peptide linker (L). Methods for making scFv molecules and designing suitable peptide linkers are described in U.S. Pat. Nos. 4,704,692; 4,946,778; Raag and Whitlow, FASEB 9:73-80 (1995) and Bird and Walker, TIBTECH, 9:132-137, 1991.

Techniques for producing single domain antibodies (DABs or VHH) are also known in the art, as disclosed for example in Cossins et al. (Prot Express Purif 51:253-259, 2006). Single domain antibodies may be obtained, for example, from camels, alpacas or llamas by standard immunization techniques. (See, e.g., Muyldermans et al., TIES 26:230-235, 2001; Yau et al., J Immunol Methods 281:161-75, 2003; Maass et al., J Immunol Methods 324:13-25, 2007). The VHH may have potent antigen-binding capacity and can interact with novel epitopes that are inaccessible to conventional VH-VL pairs. (Muyldermans et al., 2001). Alpaca serum IgG contains about 50% camelid heavy chain only IgG antibodies (HCAbs) (Maass et al., 2007). Alpacas may be immunized with known antigens, such as TNF-α, and VHHs can be isolated that bind to and neutralize the target antigen (Maass et al., 2007). PCR primers that amplify virtually all alpaca VHH coding sequences have been identified and may be used to construct alpaca VHH phage display libraries, which can be used for antibody fragment isolation by standard biopanning techniques well known in the art (Maass et al., 2007). In certain embodiments, anti-pancreatic cancer VHH antibody fragments may be utilized in the claimed compositions and methods.

An antibody fragment can be prepared by proteolytic hydrolysis of the full length antibody or by expression in E. coli or another host of the DNA coding for the fragment. An antibody fragment can be obtained by pepsin or papain digestion of full length antibodies by conventional methods. These methods are described, for example, by Goldenberg, U.S. Pat. Nos. 4,036,945 and 4,331,647 and references contained therein. Also, see Nisonoff et al., Arch Biochem. Biophys. 89: 230 (1960); Porter, Biochem. J. 73: 119 (1959), Edelman et al., in Meth Enzymol Vol. 1, page 422 (Academic Press, 1967), and Coligan at pages 2.8.1-2.8.10 and 2.10.-2.10.4.

Target Antigens and Exemplary Antibodies

In one embodiment, antibodies are used that recognize and/or bind to antigens that are expressed at high levels on target cells and that are expressed predominantly or exclusively on diseased cells versus normal tissues. Exemplary antibodies of use for therapy of, for example, cancer include but are not limited to LL1 (anti-CD74), LL2 or RFB4 (anti-CD22), veltuzumab (hA20, anti-CD20), rituxumab (anti-CD20), obinutuzumab (GA101, anti-CD20), lambrolizumab (anti-PD1), nivolumab (anti-PD1), MK-3475 (anti-PD1), AMP-224 (anti-PD1), pidilizumab (anti-PD1), MDX-1105 (anti-PD-L1), MEDI4736 (anti-PD-L1), MPDL3280A (anti-PD-L1), BMS-936559 (anti-PD-L1), ipilimumab (anti-CTLA4), trevilizumab (anti-CTL4A), RS7 (anti-epithelial glycoprotein-1 (EGP-1, also known as TROP-2)), PAM4 or KC4 (both anti-mucin), MN-14 (anti-carcinoembryonic antigen (CEA, also known as CD66e or CEACAM-5), MN-15 or MN-3 (anti-CEACAM-6), Mu-9 (anti-colon-specific antigen-p), Immu 31 (an anti-alpha-fetoprotein), R1 (anti-IGF-1R), A19 (anti-CD19), TAG-72 (e.g., CC49), Tn, J591 or HuJ591 (anti-PSMA (prostate-specific membrane antigen)), AB-PG1-XG1-026 (anti-PSMA dimer), D2/B (anti-PSMA), G250 (an anti-carbonic anhydrase IX MAb), L243 (anti-HLA-DR) alemtuzumab (anti-CD52), bevacizumab (anti-VEGF), cetuximab (anti-EGFR), gemtuzumab (anti-CD33), ibritumomab tiuxetan (anti-CD20); panitumumab (anti-EGFR); tositumomab (anti-CD20); PAM4 (aka clivatuzumab, anti-mucin), BWA-3 (anti-histone H2A/H4), LG2-1 (anti-histone H3), MRA12 (anti-histone H1), PR1-1 (anti-histone H2B), LG11-2 (anti-histone H2B), LG2-2 (anti-histone H2B), and trastuzumab (anti-ErbB2). Such antibodies are known in the art (e.g., U.S. Pat. Nos. 5,686,072; 5,874,540; 6,107,090; 6,183,744; 6,306,393; 6,653,104; 6,730.300; 6,899,864; 6,926,893; 6,962,702; 7,074,403; 7,230,084; 7,238,785; 7,238,786; 7,256,004; 7,282,567; 7,300,655; 7,312,318; 7,585,491; 7,612,180; 7,642,239; and U.S. Patent Application Publ. No. 20050271671; 20060193865; 20060210475; 20070087001.) Specific known antibodies of use include hPAM4 (U.S. Pat. No. 7,282,567), hA20 (U.S. Pat. No. 7,151,164), hA19 (U.S. Pat. No. 7,109,304), hIMMU-31 (U.S. Pat. No. 7,300,655), hLL1 (U.S. Pat. No. 7,312,318), hLL2 (U.S. Pat. No. 5,789,554), hMu-9 (U.S. Pat. No. 7,387,773), hL243 (U.S. Pat. No. 7,612,180), hMN-14 (U.S. Pat. No. 6,676,924), hMN-15 (U.S. Pat. No. 8,287,865), hR1 (U.S. patent application Ser. No. 13/688,812), hRS7 (U.S. Pat. No. 7,238,785), hMN-3 (U.S. Pat. No. 7,541,440), AB-PG1-XG1-026 (U.S. patent application Ser. No. 11/983,372, deposited as ATCC PTA-4405 and PTA-4406) and D2/B (WO 2009/130575).

Other useful antigens that may be targeted using the described conjugates include carbonic anhydrase IX, B7, CCL19, CCL21, CSAp, HER-2/neu, BrE3, CD1, CD1a, CD2, CD3, CD4, CD S, CD8, CD11A, CD14, CD15, CD16, CD18, CD19, CD20 (e.g., C2B8, hA20, 1F5 MAbs), CD21, CD22, CD23, CD25, CD29, CD30, CD32b, CD33, CD37, CD38, CD40, CD40L, CD44, CD45, CD46, CD52, CD54, CD55, CD59, CD64, CD67, CD70, CD74, CD79a, CD80, CD83, CD95, CD126, CD133, CD138, CD147, CD154, CEACAM-5, CEACAM-6, CTLA4, alpha-fetoprotein (AFP), VEGF (e.g., AVASTIN®, fibronectin splice variant), ED-B fibronectin (e.g., L19), EGP-1 (TROP-2), EGP-2 (e.g., 17-1A), EGF receptor (ErbB1) (e.g., ERBITUX®), ErbB2, ErbB3, Factor H, FHL-1, Flt-3, folate receptor, Ga 733, GRO-β, HMGB-1, hypoxia inducible factor (HIF), HM1.24, HER-2/neu, insulin-like growth factor (ILGF), IFN-γ, IFN-α, IFN-β, IFN-k, IL-2R, IL-4R, IL-6R, IL-13R, IL-15R, IL-17R, IL-18R, IL-2, IL-6, IL-8, IL-12, IL-15, IL-17, IL-18, IL-25, IP-10, IGF-1R, Ia, HM1.24, gangliosides, HCG, the HLA-DR antigen to which L243 binds, CD66 antigens, i.e., CD66a-d or a combination thereof, MAGE, mCRP, MCP-1, MIP-1A, MIP-1B, macrophage migration-inhibitory factor (MIF), MUC1, MUC2, MUC3, MUC4, MUC5ac, placental growth factor (PlGF), PSA (prostate-specific antigen), PSMA, PAM4 antigen, PD1 receptor, NCA-95, NCA-90, A3, A33, Ep-CAM, KS-1, Le(y), mesothelin, S100, tenascin, TAC, Tn antigen, Thomas-Friedenreich antigens, tumor necrosis antigens, tumor angiogenesis antigens, TNF-α, TRAIL receptor (R1 and R2), TROP-2, VEGFR, RANTES, T101, as well as cancer stem cell antigens, complement factors C3, C3a, C3b, C5a, C5, and an oncogene product.

A comprehensive analysis of suitable antigen (Cluster Designation, or CD) targets on hematopoietic malignant cells, as shown by flow cytometry and which can be a guide to selecting suitable antibodies for immunotherapy, is Craig and Foon, Blood 111(8):3941-3967, 2008.

The CD66 antigens consist of five different glycoproteins with similar structures, CD66a-e, encoded by the carcinoembryonic antigen (CEA) gene family members, BCG, CGM6, NCA, CGM1 and CEA, respectively. These CD66 antigens (e.g., CEACAM-6) are expressed mainly in granulocytes, normal epithelial cells of the digestive tract and tumor cells of various tissues. Also included as suitable targets for cancers are cancer testis antigens, such as NY-ESO-1 (Theurillat et al., Int. J. Cancer 2007; 120(11):2411-7), as well as CD79a in myeloid leukemia (Kozlov et al., Cancer Genet. Cytogenet. 2005; 163(1):62-7) and also B-cell diseases, and CD79b for non-Hodgkin's lymphoma (Poison et al., Blood 110(2):616-623). A number of the aforementioned antigens are disclosed in U.S. Pat. Applic. Pub. 2004/0166115. Cancer stem cells, which are ascribed to be more therapy-resistant precursor malignant cell populations (Hill and Perris, J. Natl. Cancer Inst. 2007; 99:1435-40), have antigens that can be targeted in certain cancer types, such as CD133 in prostate cancer (Maitland et al., Ernst Schering Found. Sympos. Proc. 2006; 5:155-79), non-small-cell lung cancer (Donnenberg et al., J. Control Release 2007; 122(3):385-91), and glioblastoma (Beier et al., Cancer Res. 2007; 67(9):4010-5), and CD44 in colorectal cancer (Dalerba er al., Proc. Natl. Acad. Sci. USA 2007; 104(24)10158-63), pancreatic cancer (Li et al., Cancer Res. 2007; 67(3): 1030-7), and in head and neck squamous cell carcinoma (Prince et al., Proc. Natl. Acad. Sci. USA 2007; 104(3)973-8).

Anti-cancer antibodies have been demonstrated to bind to histones in some case. Kato et al. (1991, Hum Antibodies Hybridomas 2:94-101) reported that the lung cancer-specific human monoclonal antibody HB4C5 binds to histone H2B. Garzelli et al. (Immunol Lett 39:277-282, 1994) observed that Epstein-Barr virus-transformed human B lymphocytes produce natural antibodies to histones. In certain embodiments, antibodies against histones may be of use in the subject combinations. Known anti-histone antibodies include, but are not limited to, BWA-3 (anti-histone H2A/H4), LG2-1 (anti-histone H3), MRA12 (anti-histone H1), PR1-1 (anti-histone H2B), LG11-2 (anti-histone H2B), and LG2-2 (anti-histone H2B) (see, e.g., Monestier et al., 1991, Eur J Immunol 21:1725-31; Monestier et al., 1993, Molec Immunol 30:1069-75).

For multiple myeloma therapy, suitable targeting antibodies have been described against, for example, CD38 and CD138 (Stevenson, Mol Med 2006; 12(11-12):345-346; Tassone et al., Blood 2004; 104(12):3688-96), CD74 (Stein et al., ibid.), CS1 (Tai et al., Blood 2008; 112(4):1329-37, and CD40 (Tai et al., 2005; Cancer Res. 65(13):5898-5906).

Macrophage migration inhibitory factor (MIF) is an important regulator of innate and adaptive immunity and apoptosis. It has been reported that CD74 is the endogenous receptor for MIF (Leng et al., 2003, J Exp Med 197:1467-76). The therapeutic effect of antagonistic anti-CD74 antibodies on MIF-mediated intracellular pathways may be of use for treatment of a broad range of disease states, such as cancers of the bladder, prostate, breast, lung, colon and chronic lymphocytic leukemia (e.g., Meyer-Siegler et al., 2004, BMC Cancer 12:34; Shachar & Haran, 2011, Leuk Lymphoma 52:1446-54). Milatuzumab (hLL1) is an exemplary anti-CD74 antibody of therapeutic use for treatment of MIF-mediated diseases.

An example of an antibody/antigen pair is LL1, an anti-CD74 MAb (invariant chain, class II-specific chaperone, Ii) (see, e.g., U.S. Pat. Nos. 6,653,104; 7,312,318. The CD74 antigen is highly expressed on B-cell lymphomas (including multiple myeloma) and leukemias, certain T-cell lymphomas, melanomas, colonic, lung, and renal cancers, glioblastomas, and certain other cancers (Ong et al., Immunology 98:296-302 (1999)). A review of the use of CD74 antibodies in cancer is contained in Stein et al., Clin Cancer Res. 2007 Sep. 15; 13(18 Pt 2):55565-5563s. The diseases that are preferably treated with anti-CD74 antibodies include, but are not limited to, non-Hodgkin's lymphoma, Hodgkin's disease, melanoma, lung, renal, colonic cancers, glioblastome multiforme, histiocytomas, myeloid leukemias, and multiple myeloma.

In another preferred embodiment, the therapeutic combinations can be used against pathogens, since antibodies against pathogens are known. For example, antibodies and antibody fragments which specifically bind markers produced by or associated with infectious lesions, including viral, bacterial, fungal and parasitic infections, for example caused by pathogens such as bacteria, rickettsia, mycoplasma, protozoa, fungi, and viruses, and antigens and products associated with such microorganisms have been disclosed, inter alia, in Hansen et al., U.S. Pat. No. 3,927,193 and Goldenberg U.S. Pat. Nos. 4,331,647, 4,348,376, 4,361,544, 4,468,457, 4,444,744, 4,818,709 and 4,624,846, and in Reichert and Dewitz (Nat Rev Drug Discovery 2006; 5:191-195). A review listing antibodies against infectious organisms (antitoxin and antiviral antibodies), as well as other targets, is contained in Casadevall, Clin Immunol 1999; 93(1):5-15. Commercially antibodies (e.g., KPL, Inc., Gaithersburg, Md.) are available against a wide variety of human pathogens.

In various embodiments, the claimed methods and compositions may utilize any of a variety of antibodies known in the art (or fragments thereof). Antibodies of use may be commercially obtained from a number of known sources. The antigen binding domains of the cloned antibodies may be amplified, excised, ligated into an expression vector, transfected into an adapted host cell and used for protein production, using standard techniques well known in the art (see, e.g., U.S. Pat. Nos. 7,531,327; 7,537,930; 7,608,425 and 7,785,880).

In other embodiments, the antibody complexes bind to a WIC class I, WIC class II or accessory molecule, such as CD40, CD54, CD80 or CD86. The antibody complex also may bind to a leukocyte activation cytokine, or to a cytokine mediator, such as NF-κB.

In certain embodiments, one of the two different targets may be a cancer cell receptor or cancer-associated antigen, particularly one that is selected from the group consisting of B-cell lineage antigens (CD19, CD20, CD21, CD22, CD23, etc.), VEGF, VEGFR, EGFR, carcinoembryonic antigen (CEA), placental growth factor (PlGF), tenascin, HER-2/neu, EGP-1, EGP-2, CD25, CD30, CD33, CD38, CD40, CD45, CD52, CD74, CD80, CD138, NCA66, CEACAM-1, CEACAM-5, CEACAM-6 (carcinoembryonic antigen-related cellular adhesion molecule 6), MUC1, MUC2, MUC3, MUC4, MUC16, IL-6, α-fetoprotein (AFP), A3, CA125, colon-specific antigen-p (CSAp), folate receptor, HLA-DR, human chorionic gonadotropin (HCG), Ia, EL-2, insulin-like growth factor (IGF) and IGF receptor, KS-1, Le(y), MAGE, necrosis antigens, PAM-4, prostatic acid phosphatase (PAP), Prl, prostate specific antigen (PSA), prostate specific membrane antigen (PSMA), S100, T101, TAC, TAG72, TRAIL receptors, and carbonic anhydrase IX.

Other antibodies that may be used include antibodies against infectious disease agents, such as bacteria, viruses, mycoplasms or other pathogens. Many antibodies against such infectious agents are known in the art and any such known antibody may be used in the claimed methods and compositions.

Immunoconjugates

In certain embodiments, antibodies or fragments thereof may be conjugated to one or more therapeutic or diagnostic agents. The therapeutic agents can be different, e.g. a drug and a radioisotope. Therapeutic and diagnostic agents also can be attached, for example to reduced SH groups and/or to carbohydrate side chains. Many methods for making covalent or non-covalent conjugates of therapeutic or diagnostic agents with antibodies or fusion proteins are known in the art.

Therapeutic Agents

In alternative embodiments, therapeutic agents such as cytotoxic agents, anti-angiogenic agents, pro-apoptotic agents, antibiotics, hormones, hormone antagonists, chemokines, drugs, prodrugs, toxins, enzymes or other agents may be used, either conjugated to the CAR or PMA and/or other antibodies or separately administered. Drugs of use may possess a pharmaceutical property selected from the group consisting of antimitotic, antikinase, alkylating, antimetabolite, antibiotic, alkaloid, anti-angiogenic, pro-apoptotic agents and combinations thereof.

Methods of Treatment

The CAR system of the present invention can be used in the treatment of a subject having a disease such as cancer. The methods of treatment encompassed by the invention generally includes the steps of (i) obtaining a population of autologous or heterologous cytotoxic lymphocytes, (ii) culturing the lymphocytes under conditions that promote the activation of the cells, (iii) transfecting the lymphocytes with an expression vector encoding a CAR, (iv) administering a formulation comprising the transfected lymphocytes to a subject having cancer, and (v) administering a formulation comprising PMA to the subject.

According to one embodiment, the present invention is used for therapy of cancer. Examples of cancers include, but are not limited to, carcinoma, lymphoma, glioblastoma, melanoma, sarcoma, and leukemia, myeloma, or lymphoid malignancies. More particular examples of such cancers are noted below and include: squamous cell cancer (e.g., epithelial squamous cell cancer), Ewing sarcoma, Wilms tumor, astrocytomas, lung cancer including small-cell lung cancer, non-small-cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma multiforme, cervical cancer, ovarian cancer, liver cancer, bladder cancer, hepatoma, hepatocellular carcinoma, neuroendocrine tumors, medullary thyroid cancer, differentiated thyroid carcinoma, breast cancer, ovarian cancer, colon cancer, rectal cancer, endometrial or uterine carcinoma, salivary gland carcinoma, kidney or renal cancer, prostate cancer, vulvar cancer, anal carcinoma, penile carcinoma, as well as head-and-neck cancer. The term “cancer” includes primary malignant cells or tumors (e.g., those whose cells have not migrated to sites in the subject's body other than the site of the original malignancy or tumor) and secondary malignant cells or tumors (e.g., those arising from metastasis, the migration of malignant cells or tumor cells to secondary sites that are different from the site of the original tumor).

Other examples of cancers or malignancies include, but are not limited to: Acute Childhood Lymphoblastic Leukemia, Acute Lymphoblastic Leukemia, Acute Lymphocytic Leukemia, Acute Myeloid Leukemia, Adrenocortical Carcinoma, Adult (Primary) Hepatocellular Cancer, Adult (Primary) Liver Cancer, Adult Acute Lymphocytic Leukemia, Adult Acute Myeloid Leukemia, Adult Hodgkin's Lymphoma, Adult Lymphocytic Leukemia, Adult Non-Hodgkin's Lymphoma, Adult Primary Liver Cancer, Adult Soft Tissue Sarcoma, AIDS-Related Lymphoma, AIDS-Related Malignancies, Anal Cancer, Astrocytoma, Bile Duct Cancer, Bladder Cancer, Bone Cancer, Brain Stem Glioma, Brain Tumors, Breast Cancer, Cancer of the Renal Pelvis and Ureter, Central Nervous System (Primary) Lymphoma, Central Nervous System Lymphoma, Cerebellar Astrocytoma, Cerebral Astrocytoma, Cervical Cancer, Childhood (Primary) Hepatocellular Cancer, Childhood (Primary) Liver Cancer, Childhood Acute Lymphoblastic Leukemia, Childhood Acute Myeloid Leukemia, Childhood Brain Stem Glioma, Childhood Cerebellar Astrocytoma, Childhood Cerebral Astrocytoma, Childhood Extracranial Germ Cell Tumors, Childhood Hodgkin's Disease, Childhood Hodgkin's Lymphoma, Childhood Hypothalamic and Visual Pathway Glioma, Childhood Lymphoblastic Leukemia, Childhood Medulloblastoma, Childhood Non-Hodgkin's Lymphoma, Childhood Pineal and Supratentorial Primitive Neuroectodermal Tumors, Childhood Primary Liver Cancer, Childhood Rhabdomyosarcoma, Childhood Soft Tissue Sarcoma, Childhood Visual Pathway and Hypothalamic Glioma, Chronic Lymphocytic Leukemia, Chronic Myelogenous Leukemia, Colon Cancer, Cutaneous T-Cell Lymphoma, Endocrine Pancreas Islet Cell Carcinoma, Endometrial Cancer, Ependymoma, Epithelial Cancer, Esophageal Cancer, Ewing's Sarcoma and Related Tumors, Exocrine Pancreatic Cancer, Extracranial Germ Cell Tumor, Extragonadal Germ Cell Tumor, Extrahepatic Bile Duct Cancer, Eye Cancer, Breast Cancer, Gaucher's Disease, Gallbladder Cancer, Gastric Cancer, Gastrointestinal Carcinoid Tumor, Gastrointestinal Tumors, Germ Cell Tumors, Gestational TROPhoblastic Tumor, Hairy Cell Leukemia, Head and Neck Cancer, Hepatocellular Cancer, Hodgkin's Lymphoma, Hypergammaglobulinemia, Hypopharyngeal Cancer, Intestinal Cancers, Intraocular Melanoma, Islet Cell Carcinoma, Islet Cell Pancreatic Cancer, Kaposi's Sarcoma, Kidney Cancer, Laryngeal Cancer, Lip and Oral Cavity Cancer, Liver Cancer, Lung Cancer, Lymphoproliferative Disorders, Macroglobulinemia, Malignant Mesothelioma, Malignant Thymoma, Medulloblastoma, Melanoma, Mesothelioma, Metastatic Occult Primary Squamous Neck Cancer, Metastatic Primary Squamous Neck Cancer, Metastatic Squamous Neck Cancer, Multiple Myeloma, Multiple Myeloma/Plasma Cell Neoplasm, Myelodysplastic Syndrome, Myelogenous Leukemia, Myeloid Leukemia, Myeloproliferative Disorders, Nasal Cavity and Paranasal Sinus Cancer, Nasopharyngeal Cancer, Neuroblastoma, Non-Hodgkin's Lymphoma, Nonmelanoma Skin Cancer, Non-Small Cell Lung Cancer, Occult Primary Metastatic Squamous Neck Cancer, Oropharyngeal Cancer, Osteo-/Malignant Fibrous Sarcoma, Osteosarcoma/Malignant Fibrous Histiocytoma, Osteosarcoma/Malignant Fibrous Histiocytoma of Bone, Ovarian Epithelial Cancer, Ovarian Germ Cell Tumor, Ovarian Low Malignant Potential Tumor, Pancreatic Cancer, Paraproteinemias, Polycythemia vera, Parathyroid Cancer, Penile Cancer, Pheochromocytoma, Pituitary Tumor, Primary Central Nervous System Lymphoma, Primary Liver Cancer, Prostate Cancer, Rectal Cancer, Renal Cell Cancer, Renal Pelvis and Ureter Cancer, Retinoblastoma, Rhabdomyosarcoma, Salivary Gland Cancer, Sarcoidosis Sarcomas, Sezary Syndrome, Skin Cancer, Small Cell Lung Cancer, Small Intestine Cancer, Soft Tissue Sarcoma, Squamous Neck Cancer, Stomach Cancer, Supratentorial Primitive Neuroectodermal and Pineal Tumors, T-Cell Lymphoma, Testicular Cancer, Thymoma, Thyroid Cancer, Transitional Cell Cancer of the Renal Pelvis and Ureter, Transitional Renal Pelvis and Ureter Cancer, Trophoblastic Tumors, Ureter and Renal Pelvis Cell Cancer, Urothelial Cancer, Uterine Cancer, Uterine Sarcoma, Vaginal Cancer, Visual Pathway and Hypothalamic Glioma, Vulvar Cancer, Waldenstrom's Macroglobulinemia, Wilms' Tumor, and any other hyperproliferative disease, besides neoplasia, located in an organ system listed above.

The methods and compositions described and claimed herein may be used to treat malignant or premalignant conditions and to prevent progression to a neoplastic or malignant state, including but not limited to those disorders described above. Such uses are indicated in conditions known or suspected of preceding progression to neoplasia or cancer, in particular, where non-neoplastic cell growth consisting of hyperplasia, metaplasia, or most particularly, dysplasia has occurred (for review of such abnormal growth conditions, see Robbins and Angell, Basic Pathology, 2d Ed., W. B. Saunders Co., Philadelphia, pp. 68-79 (1976)).

Additional pre-neoplastic disorders which can be treated include, but are not limited to, benign dysproliferative disorders (e.g., benign tumors, fibrocystic conditions, tissue hypertrophy, intestinal polyps or adenomas, and esophageal dysplasia), leukoplakia, keratoses, Bowen's disease, Farmer's Skin, solar cheilitis, and solar keratosis.

In preferred embodiments, the method of the invention is used to inhibit growth, progression, and/or metastasis of cancers, in particular those listed above.

Additional hyperproliferative diseases, disorders, and/or conditions include, but are not limited to, progression, and/or metastases of malignancies and related disorders such as leukemia (including acute leukemias (e.g., acute lymphocytic leukemia, acute myelocytic leukemia (including myeloblastic, promyelocytic, myelomonocytic, monocytic, and erythroleukemia)) and chronic leukemias (e.g., chronic myelocytic (granulocytic) leukemia and chronic lymphocytic leukemia)), polycythemia vera, lymphomas (e.g., Hodgkin's disease and non-Hodgkin's disease), multiple myeloma, Waldenstrom's macroglobulinemia, heavy chain disease, and solid tumors including, but not limited to, sarcomas and carcinomas such as fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, emangioblastoma, acoustic neuroma, oligodendroglioma, meningioma, melanoma, neuroblastoma, and retinoblastoma.

The invention also includes variations on this theme such, as administering the formulation comprising one or more PMAs (i.e., PMAs having different targeting moieties) to the subject before the formulation comprising the transfected lymphocytes, or at the same time as the formulation comprising the transfected lymphocytes. A further variation includes culturing the formulation comprising the transfected lymphocytes with the PMA prior to administration to the subject.

The population of cytotoxic lymphocytes can be obtained from a subject by means well known in the art. For example, cytotoxic T cells can be obtained by collecting peripheral blood from the subject, subjecting the blood to Ficoll density gradient centrifugation, and then using a negative T cell isolation kit (such as EasySep™ T Cell Isolation Kit) to isolate a population of cytotoxic T cells from the blood. While the population of cytotoxic lymphocytes need not be pure and may contain other blood cells such as T cells, monocytes, macrophages, natural killer cells and B cells, depending of the population being collected, preferably the population comprises at least about 90% of the selected cell type. In particular aspects, the population comprises at least about 90%, 91%, 92%, 93%, 94%, 95%, 96%, 97%, 98%, 99%, or even 100% of the selected cell type. As indicated earlier, the population of cells may come from the subject to be treated, from one or more different subjects, or the population may be a combination of cells from the subject to be treated and one or more different subjects.

After the population of cytotoxic lymphocytes is obtained, the cells are cultured under conditions that promote the activation of the cells. The culture conditions will be such that the cells can be administered to a subject without concern for reactivity against components of the culture. For example, when the population will be administered to a human, the culture conditions will not include bovine serum products, such as bovine serum albumin. The activation of the lymphocytes in the culture can be achieved by introducing known activators into the culture, such as anti-CD3 antibodies in the case of cytotoxic T cells. Other suitable activators include anti-CD28 antibodies. The population of lymphocytes will generally be cultured under conditions promoting activation for about 1 to 4 days. The appropriate level of cellular activation can be determined by cell size, proliferation rate or activation markers by flow cytometry.

After the population of cytotoxic lymphocytes has been cultured under conditions promoting activation, the cells are transfected with an expression vector encoding a CAR. Such vectors are described above, along with suitable means of transfection. After transfection, the resulting population of cells can be immediately administered to a subject or the cells can be culture for at least about 1, 2, 3, 4, 5, 6, 7, 8, 9, 10, 11, 12, 13, 14, 15, 16, 17, 18 or more days, or between about 5 and 12 days, between about 6 and 13 days, between about 7 and 14 days, or between about 8 and 15 days, for example, to allow time for the cells to recover from the transfection. Suitable culture conditions with be the same as those conditions under which the cells were culture while activation was being promoted, either with or without the agent that was used to promote activation and expansion.

When the transfected cells are ready a formulation comprising the cells is prepared and administered to a subject having cancer. Prior to administration, the population of cells can be washed and resuspended in a pharmaceutically acceptable carrier or diluent to form the formulation. Such carriers and diluents include, but are not limited to, sterile 290 mOsm saline, infusible cryomedia (containing Plasma-Lyte A, dextrose, sodium chloride injection, human serum albumin and DMSO), 0.9% NaCl with 2% human serum albumin or any other sterile 290 mOsm infusible materials. Alternatively, depending on the identity of the culture media used in the previous step, the cells can be administered in the culture media as the formulation, or concentrated and resuspended in the culture media before administration. The formulation can be administered to the subject via suitable means, such as parenteral administration, e.g., intradermally, subcutaneously, intramuscularly, intraperitoneally, intravenously, or intrathecally.

The total number of cells and the concentration of cells in the formulation administered to a subject will vary depending on a number of factors including the type of cytotoxic lymphocytes being used, the binding specificity of the CAR, the identity of the targeted moiety and the ligand, the identity of the cancer or tumor to be treated, the location in the subject of the cancer or tumor, the means used to administer the formulations to the subject, and the health, age and weight of the subject being treated. However, suitable formulations comprising transduced lymphocytes include those having a volume of between about 5 ml and 200 ml, containing from about 1×10⁵ to 1×10¹⁵ transduced cells. Typical formulations comprise a volume of between about 10 ml and 125 ml, containing from about 1×10⁷ to 1×10¹⁰ transduced cells. An exemplary formulation comprises about 1×10⁹ transduced cells in a volume of about 100 ml.

The final step in the method is the administration of a formulation comprising PMA to the subject. As described above, the PMA will be prepared in a formulation appropriate for the subject receiving the molecules. The concentration of PMA in a PMA formulation will vary depending on factors that include the binding specificity of the CAR, the identity of the targeted moiety and the ligand, the identity of the cancer or tumor to be treated, the location in the subject of the cancer or tumor, the means used to administer the formulations to the subject, and the health, age and weight of the subject being treated. However, suitable formulations comprising PMA include those having a volume of between about 1 ml and 50 ml and contain between about 20 ug/kg body weight and 3 mg/kg body weight PMA. Typical formulations comprise a volume of between about 5 ml and 20 ml and contain between about 0.2 mg/kg body weight and 0.4 mg/kg body weight PMA. An exemplary formulation comprises about 50 ug/kg body weight PMA in a volume of about 10 ml.

The timing between the administration of transduced lymphocyte formulation and the PMA formation may range widely depending on factors that include the type of cytotoxic lymphocytes being used, the binding specificity of the CAR, the identity of the targeted moiety and the ligand, the identity of the cancer or tumor to be treated, the location in the subject of the cancer or tumor, the means used to administer the formulations to the subject, and the health, age and weight of the subject being treated. Indeed, the PMA formation may be administered prior to, simultaneous with, or after the lymphocyte formulation. In general, the PMA formation will be administered after the lymphocyte formulation, such as within 3, 6, 9, 12, 15, 18, 21, or 24 hours, or within 0.5, 1, 1.5, 2, 2.5, 3, 4 5, 6, 7, 8, 9, 10 or more days. When the PMA formation is administered before the lymphocyte formulation, the lymphocyte formulation will generally be administered within about 0.25, 0.5, 0.75, 1, 2, 3, 4, 5, 6, 7, 8, 9, 10, 11, 12 or more hours. When the PMA formation and the lymphocyte formulation are added simultaneously, it is preferable that the formations are not combined and thus administered separately to the subject.

Depending on the cancer being treatment the step of administering the lymphocyte formulation, or the step of administering the PMA formulation, or both, can be repeated one or more times. The particular number and order of the steps is not limited as the attending physician may find that a method can be practiced to the advantage of the subject using one or more of the following methodologies, or others not named here: (i) administering the lymphocyte formulation (A) followed by the PMA formulation (B), i.e., A then B; (ii) B then A; (iii) A then B then A then B; (iv) A then B then A; (v) B then A then B then A; (vi) A then A then B; (vii) B then A then A; (vii) B then B then A.

The formulations can be administered as single continuous doses, or they can be divided and administered as a multiple-dose regimen depending on the reaction (i.e., side effects) of the patient to the formulations.

The efficacy of immune system induction for cancer therapy may be enhanced by combination with other agents that, for example, reduce tumor burden prior to administration of CAR-T or CAR-NK. Antibody-drug conjugates (ADCs) can effectively reduce tumor burden in many types of cancers, as documented by pathological complete response (pCR) in neoadjuvant therapy of TNBC. Numerous exemplary ADCs are known in the art, such as IMMU-130 (labetuzumab-SN-38), IMMU-132 (hRS7-SN-38) and milatuzumab-doxorubicin or antibody conjugates of pro-2-pyrrolinodoxorubicin (Pro2PDox), as discussed below. Other exemplary ADCs of use may include gemtuzumab ozogamicin for AML (subsequently withdrawn from the market), brentuximab vedotin for ALCL and Hodgkin lymphoma, and trastuzumab emtansine for HER2-positive metastatic breast cancer (Verma et al., N Engl J Med 367:1783-91, 2012; Bross et al., Clin Cancer Res 7:1490-96, 2001; Francisco et al., Blood 102:1458-65, 2003). Numerous other candidate ADCs are currently in clinical testing, such as inotuzumab ozogamicin (Pfizer), glembatumomab vedotin (Celldex Therapeutics), SAR3419 (Sanofi-Aventis), SAR56658 (Sanofi-Aventis), AMG-172 (Amgen), AMG-595 (Amgen), BAY-94-9343 (Bayer), BIIB015 (Biogen Idec), BT062 (Biotest), SGN-75 (Seattle Genetics), SGN-CD19A (Seattle Genetics), vorsetuzumab mafodotin (Seattle Genetics), ABT-414 (AbbVie), ASG-5ME (Agensys), ASG-22ME (Agensys), ASG-16M8F (Agensys), IMGN-529 (ImmunoGen), IMGN-853 (ImmunoGen), MDX-1203 (Medarex), MLN-0264 (Millenium), RG-7450 (Roche/Genentech), RG-7458 (Roche/Genentech), RG-7593 (Roche/Genentech), RG-7596 (Roche/Genentech), RG-7598 (Roche/Genentech), RG-7599 (Roche/Genentech), RG-7600 (Roche/Genentech), RG-7636 (Roche/Genentech), anti-PSMA ADC (Progenics), lorvotuzumab mertansine (ImmunoGen), milatuzumab-doxorubicin (Immunomedics), IMMU-130 (Immunomedics) and IMMU-132 (Immunomedics). (See, e.g., Li et al., Drug Disc Ther 7:178-84, 2013; Firer & Gellerman, J Hematol Oncol 5:70, 2012; Beck et al., Discov Med 10:329-39, 2010; Mullard, Nature Rev Drug Discovery 12:329, 2013.) Any such known ADC may be used in combination with a CAR-T or CAR-NK construct as described herein. Preferably, where an ADC is used in combination with a CAR-T or CAR-NK, the ADC is administered prior to the CAR-T or CAR-NK.

Combination therapy with immunostimulatory antibodies may enhance efficacy, for example against tumor cells. Morales-Kastresana et al. (Clin Cancer Res 19:6151-62, 2013) showed that the combination of anti-PD-L1 (10B5) antibody with anti-CD137 (1D8) and anti-OX40 (OX86) antibodies provided enhanced efficacy in a transgenic mouse model of hepatocellular carcinoma. Combination of anti-CTLA4 and anti-PD1 antibodies has also been reported to be highly efficacious (Wolchok et al., N Engl J Med 369:122-33, 2013). Combination of rituximab with anti-KIR antibody, such as lirlumab (Innate Pharma) or IPH2101 (Innate Pharma), was also more efficacious against hematopoietic tumors (Kohrt et al., 2012). Combination therapy may include combinations with multiple antibodies that are immunostimulatory, anti-tumor or anti-infectious agents.

Alternative antibodies that may be used for treatment of various disease states include, but are not limited to, abciximab (anti-glycoprotein IIb/IIIa), alemtuzumab (anti-CD52), bevacizumab (anti-VEGF), cetuximab (anti-EGFR), gemtuzumab (anti-CD33), ibritumomab (anti-CD20), panitumumab (anti-EGFR), rituximab (anti-CD20), tositumomab (anti-CD20), trastuzumab (anti-ErbB2), lambrolizumab (anti-PD1 receptor), nivolumab (anti-PD1 receptor), ipilimumab (anti-CTLA4), abagovomab (anti-CA-125), adecatumumab (anti-EpCAM), atlizumab (anti-IL-6 receptor), benralizumab (anti-CD125), obinutuzumab (GA101, anti-CD20), CC49 (anti-TAG-72), AB-PG1-XG1-026 (anti-PSMA, U.S. patent application Ser. No. 11/983,372, deposited as ATCC PTA-4405 and PTA-4406), D2/B (anti-PSMA, WO 2009/130575), tocilizumab (anti-IL-6 receptor), basiliximab (anti-CD25), daclizumab (anti-CD25), efalizumab (anti-CD11a), GA101 (anti-CD20; Glycart Roche), atalizumab (anti-α4 integrin), omalizumab (anti-IgE); anti-TNF-α antibodies such as CDP571 (Ofei et al., Diabetes 45:881-85, 2011), MTNFAI, M2TNFAI, M3TNFAI, M3TNFABI, M302B, M303 (Thermo Scientific, Rockford, Ill.), infliximab (Centocor, Malvern, Pa.), certolizumab pegol (UCB, Brussels, Belgium), anti-CD40L (UCB, Brussels, Belgium), adalimumab (Abbott, Abbott Park, Ill.), belimumab (Human Genome Sciences); anti-CD38 antibodies such as MOR03087 (MorphoSys AG), MOR202 (Celgene), HuMax-CD38 (Genmab) or daratumumab (Johnson & Johnson); anti-HIV antibodies such as P4/D10 (U.S. Pat. No. 8,333,971), Ab 75, Ab 76, Ab 77 (Paulik et al., Biochem Pharmacol 58:1781-90, 1999), as well as the anti-HIV antibodies described and sold by Polymun (Vienna, Austria), also described in U.S. Pat. Nos. 5,831,034, 5,911,989, and Vcelar et al., AIDS 2007; 21(16):2161-2170 and Joos et al., Antimicrob. Agents Chemother. 2006; 50(5):1773-9.

In other embodiments, the CAR-T or CAR-NK therapy may be of use to treat subjects infected with pathogenic organisms, such as bacteria, viruses or fungi. Exemplary fungi that may be treated include Microsporum, Trichophyton, Epidermophyton, Sporothrix schenckii, Cryptococcus neoformans, Coccidioides immitis, Histoplasma capsulatum, Blastomyces dermatitidis or Candida albican. Exemplary viruses include human immunodeficiency virus (HIV), herpes virus, cytomegalovirus, rabies virus, influenza virus, human papilloma virus, hepatitis B virus, hepatitis C virus, Sendai virus, feline leukemia virus, Reo virus, polio virus, human serum parvo-like virus, simian virus 40, respiratory syncytial virus, mouse mammary tumor virus, Varicella-Zoster virus, dengue virus, rubella virus, measles virus, adenovirus, human T-cell leukemia viruses, Epstein-Barr virus, murine leukemia virus, mumps virus, vesicular stomatitis virus, Sindbis virus, lymphocytic choriomeningitis virus or blue tongue virus. Exemplary bacteria include Bacillus anthracis, Streptococcus agalactiae, Legionella pneumophilia, Streptococcus pyogenes, Escherichia coli, Neisseria gonorrhoeae, Neisseria meningitidis, Pneumococcus spp., Hemophilis influenzae B, Treponema pallidum, Lyme disease spirochetes, Pseudomonas aeruginosa, Mycobacterium leprae, Brucella abortus, Mycobacterium tuberculosis or a Mycoplasma. Exemplary use of ADCs against infectious agents are disclosed in Johannson et al. (AIDS 20:1911-15, 2006) and Chang et al., PLos One 7:e41235, 2012).

Known antibodies against pathogens include, but are not limited to, P4D10 (anti-HIV), CR6261 (anti-influenza), exbivirumab (anti-hepatitis B), felvizumab (anti-respiratory syncytial virus), foravirumab (anti-rabies virus), motavizumab (anti-respiratory syncytial virus), palivizumab (anti-respiratory syncytial virus), panobacumab (anti-Pseudomonas), rafivirumab (anti-rabies virus), regavirumab (anti-cytomegalovirus), sevirumab (anti-cytomegalovirus), tivirumab (anti-hepatitis B), and urtoxazumab (anti-E. coli).

The subject agents may be administered in combination with one or more other immunomodulators to enhance the immune response. Immunomodulators may include, but are not limited to, a cytokine, a chemokine, a stem cell growth factor, a lymphotoxin, an hematopoietic factor, a colony stimulating factor (CSF), erythropoietin, thrombopoietin, tumor necrosis factor-α (TNF), TNF-β, granulocyte-colony stimulating factor (G-CSF), granulocyte macrophage-colony stimulating factor (GM-CSF), interferon-α, interferon-β, interferon-γ, interferon-λ, stem cell growth factor designated “S1 factor”, human growth hormone, N-methionyl human growth hormone, bovine growth hormone, parathyroid hormone, thyroxine, insulin, proinsulin, relaxin, prorelaxin, follicle stimulating hormone (FSH), thyroid stimulating hormone (TSH), luteinizing hormone (LH), hepatic growth factor, prostaglandin, fibroblast growth factor, prolactin, placental lactogen, OB protein, mullerian-inhibiting substance, mouse gonadotropin-associated peptide, inhibin, activin, vascular endothelial growth factor, integrin, NGF-β, platelet-growth factor, TGF-α, TGF-β, insulin-like growth factor-I, insulin-like growth factor-II, macrophage-CSF (M-CSF), IL-1, IL-1α, IL-2, IL-3, IL-4, IL-5, IL-6, IL-7, IL-8, IL-9, IL-10, IL-11, IL-12, IL-13, IL-14, IL-15, IL-16, IL-17, IL-18, IL-21, IL-25, LIF, FLT-3, angiostatin, thrombospondin, endostatin, or lymphotoxin.

Interferon Therapy

In various embodiments, the CAR-T or CAR-NK constructs may be used in combination with one or more interferons, such as interferon-α, interferon-β or interferon-k. Human interferons are well known in the art and the amino acid sequences of human interferons may be readily obtained from public databases. Human interferons may also be commercially obtained from a variety of vendors (e.g., Cell Signaling Technology, Inc., Danvers, Mass.; Genentech, South San Francisco, Calif.; EMD Millipore, Billerica, Mass.).

Interferon-α (IFNα) has been reported to have anti-tumor activity in animal models of cancer (Ferrantini et al., 1994, J Immunol 153:4604-15) and human cancer patients (Gutterman et al., 1980, Ann Intern Med 93:399-406). IFNα can exert a variety of direct anti-tumor effects, including down-regulation of oncogenes, up-regulation of tumor suppressors, enhancement of immune recognition via increased expression of tumor surface MHC class I proteins, potentiation of apoptosis, and sensitization to chemotherapeutic agents (Gutterman et al., 1994, PNAS USA 91:1198-205; Matarrese et al., 2002, Am J Pathol 160:1507-20; Mecchia et al., 2000, Gene Ther 7:167-79; Sabaawy et al., 1999, Int J Oncol 14:1143-51; Takaoka et al, 2003, Nature 424:516-23). For some tumors, IFNα can have a direct and potent anti-proliferative effect through activation of STAT1 (Grimley et al., 1998 Blood 91:3017-27). Interferon-α2b has been conjugated to anti-tumor antibodies, such as the hL243 anti-HLA-DR antibody and depletes lymphoma and myeloma cells in vitro and in vivo (Rossi et al., 2011, Blood 118:1877-84).

Indirectly, IFNα can inhibit angiogenesis (Sidky and Borden, 1987, Cancer Res 47:5155-61) and stimulate host immune cells, which may be vital to the overall antitumor response but has been largely under-appreciated (Belardelli et al., 1996, Immunol Today 17:369-72). IFNα has a pleiotropic influence on immune responses through effects on myeloid cells (Raefsky et al, 1985, J Immunol 135:2507-12; Luft et al, 1998, J Immunol 161:1947-53), T-cells (Carrero et al, 2006, J Exp Med 203:933-40; Pilling et al., 1999, Eur J Immunol 29:1041-50), and B-cells (Le et al, 2001, Immunity 14:461-70). As an important modulator of the innate immune system, IFNα induces the rapid differentiation and activation of dendritic cells (Belardelli et al, 2004, Cancer Res 64:6827-30; Paquette et al., 1998, J Leukoc Biol 64:358-67; Santini et al., 2000, J Exp Med 191:1777-88) and enhances the cytotoxicity, migration, cytokine production and antibody-dependent cellular cytotoxicity (ADCC) of NK cells (Biron et al., 1999, Ann Rev Immunol 17:189-220; Brunda et al. 1984, Cancer Res 44:597-601).

Interferon-β has been reported to be efficacious for therapy of a variety of solid tumors. Patients treated with 6 million units of IFN-β twice a week for 36 months showed a decreased recurrence of hepatocellular carcinoma after complete resection or ablation of the primary tumor in patients with HCV-related liver cancer (Ikeda et al., 2000, Hepatology 32:228-32). Gene therapy with interferon-0 induced apoptosis of glioma, melanoma and renal cell carcinoma (Yoshida et al., 2004, Cancer Sci 95:858-65). Endogenous IFN-0 has been observed to inhibit tumor growth by inhibiting angiogenesis in vivo (Jablonska et al., 2010, J Clin Invest. 120:1151-64.)

When used with CAR-T or CAR-NK and/or other agents, the interferon may be administered prior to, concurrently with, or after the other agent. When administered concurrently, the interferon may be either conjugated to or separate from the other agent.

Checkpoint Inhibitor Antibodies

In certain embodiments, the CAR-T or CAR-NK constructs may be utilized in combination with one or more checkpoint inhibitors, such as checkpoint inhibitor antibodies (see, e.g., Ott & Bhardwaj, 2013, Frontiers in Immunology 4:346; Menzies & Long, 2013, Ther Adv Med Oncol 5:278-85; Pardoll, 2012, Nature Reviews Cancer 12:252-64; Mavilio & Lugli, Oncoimmunology, September 1; 2(9):e26535. Epub Sep. 26, 2013).

In contrast to the majority of anti-cancer agents, checkpoint inhibitors do not target tumor cells directly, but rather target lymphocyte receptors or their ligands in order to enhance the endogenous antitumor activity of the immune system. (Pardoll, 2012, Nature Reviews Cancer 12:252-264) Because such antibodies act primarily by regulating the immune response to diseased cells, tissues or pathogens, they may be used in combination with other therapeutic modalities, such as the subject CAR-T or CAR-NK to enhance the anti-tumor effect of such agents. Because checkpoint activation may also be associated with chronic infections (Nirschl & Drake, 2013, Clin Cancer Res 19:4917-24), such combination therapies may also be of use to treat infectious disease.

Although checkpoint inhibitor antibodies against CTLA4, PD1 and PD-L1 are the most clinically advanced, other potential checkpoint antigens are known and may be used as the target of therapeutic antibodies, such as LAG3, B7-H3, B7-H4 and TIM3 (Pardoll, 2012, Nature Reviews Cancer 12:252-264).

Anti-PD1 antibodies have been used for treatment of melanoma, non-small-cell lung cancer, bladder cancer, prostate cancer, colorectal cancer, head and neck cancer, triple-negative breast cancer, leukemia, lymphoma and renal cell cancer (Topalian et al., 2012, N Engl J Med 366:2443-54; Lipson et al., 2013, Clin Cancer Res 19:462-8; Berger et al., 2008, Clin Cancer Res 14:3044-51; Gildener-Leapman et al., 2013, Oral Oncol 49:1089-96; Menzies & Long, 2013, Ther Adv Med Oncol 5:278-85). Because PD1/PD-L1 and CTLA4 act by different pathways, it is possible that combination therapy with checkpoint inhibitor antibodies against each may provide an enhanced immune response. Exemplary anti-PD1 antibodies include lambrolizumab (MK-3475, Merck), nivolumab (BMS-936558, Bristol-Myers Squibb), AMP-224 (Merck), and pidilizumab (CT-011, Curetech Ltd.). Anti-PD1 antibodies are commercially available, for example Abeam® (AB137132), Biolegend® (EH12.2H7, RMP1-14) and Affymetrix Ebioscience J105, J116, and MIH4.

Programmed cell death 1 ligand 1 (PD-L1, also known as CD274 and B7-H1) is a ligand for PD1, found on activated T cells, B cells, myeloid cells and macrophages. Although there are two endogenous ligands for PD1-PD-L1 and PD-L2, anti-tumor therapies have focused on anti-PD-L1 antibodies. The complex of PD1 and PD-L1 inhibits proliferation of CD8+ T cells and reduces the immune response (Topalian et al., 2012, N Engl J Med 366:2443-54; Brahmer et al., 2012, N Eng J Med 366:2455-65). Anti-PD-L1 antibodies have been used for treatment of non-small cell lung cancer, melanoma, colorectal cancer, renal-cell cancer, pancreatic cancer, gastric cancer, ovarian cancer, breast cancer, and hematologic malignancies (Brahmer et al., N Eng J Med 366:2455-65; Ott et al., 2013, Clin Cancer Res 19:5300-9; Radvanyi et al., 2013, Clin Cancer Res 19:5541; Menzies & Long, 2013, Ther Adv Med Oncol 5:278-85; Berger et al., 2008, Clin Cancer Res 14:13044-51). Exemplary anti-PD-L1 antibodies include MDX-1105 (MEDAREX), MEDI4736 (MEDIMMUNE) MPDL3280A (GENENTECH) and BMS-936559 (BRISTOL-MYERS SQUIBB). Anti-PD-L1 antibodies are also commercially available, for example from AFFYMETRIX EBIOSCIENCE (MIH1).

Cytotoxic T-lymphocyte antigen 4 (CTLA4, also known as CD152) is also a member of the immunoglobulin superfamily that is expressed exclusively on T-cells. CTLA4 acts to inhibit T-cell activation and is reported to inhibit helper T-cell activity and enhance regulatory T-cell immunosuppressive activity (Pardoll, 2012, Nature Reviews Cancer 12:252-264). Anti-CTL4A antibodies have been used in clinical trials for treatment of melanoma, prostate cancer, small cell lung cancer, non-small cell lung cancer (Robert & Ghiringhelli, 2009, Oncologist 14:848-61; Ott et al., 2013, Clin Cancer Res 19:5300; Weber, 2007, Oncologist 12:864-72; Wada et al., 2013, J Transl Med 11:89). Exemplary anti-CTLA4 antibodies include ipilimumab (Bristol-Myers Squibb) and tremelimumab (Pfizer). Anti-PD1 antibodies are commercially available, for example, from ABCAM® (AB134090), Sino Biological Inc. (11159-H03H, 11159-H08H), and Thermo Scientific Pierce (PA5-29572, PA5-23967, PA5-26465, MA1-12205, MA1-35914). Ipilimumab has recently received FDA approval for treatment of metastatic melanoma (Wada et al., 2013, J Transl Med 11:89).

These and other known agents that stimulate immune response to tumors and/or pathogens may be used in combination with CAR-T or CAR-NK alone or in further combination with an interferon, such as interferon-α, and/or an antibody-drug conjugate for improved cancer therapy. Other known co-stimulatory pathway modulators that may be used in combination include, but are not limited to, agatolimod, belatacept, blinatumomab, CD40 ligand, anti-B7-1 antibody, anti-B7-2 antibody, anti-B7-H4 antibody, AG4263, eritoran, anti-OX40 antibody, ISF-154, and SGN-70; B7-1, B7-2, ICAM-1, ICAM-2, ICAM-3, CD48, LFA-3, CD30 ligand, CD40 ligand, heat stable antigen, B7h, OX40 ligand, LIGHT, CD70 and CD24.

In certain embodiments, anti-KIR antibodies may also be used in combination with CAR-T or CAR-NK, interferons, ADCs and/or checkpoint inhibitor antibodies. NK cells mediate anti-tumor and anti-infectious agent activity by spontaneous cytotoxicity and by ADCC when activated by antibodies (Kohrt et al., 2014, Blood, 123: 678-86). The degree of cytotoxic response is determined by a balance of inhibitory and activating signals received by the NK cells (Kohrt et al., 2013). The killer cell immunoglobulin-like receptor (KIR) mediates an inhibitory signal that decreases NK cell response. Anti-KIR antibodies, such as lirlumab (Innate Pharma) and IPH2101 (Innate Pharma) have demonstrated anti-tumor activity in multiple myeloma (Benson et al., 2012, Blood 120:4324-33). In vitro, anti-KIR antibodies prevent the tolerogenic interaction of NK cells with target cells and augments the NK cell cytotoxic response to tumor cells (Kohrt et al., 2014, Blood, 123: 678-86). In vivo, in combination with rituximab (anti-CD20), anti-KIR antibodies at a dose of 0.5 mg/kg induced enhanced NK cell-mediated, rituximab-dependent cytotoxicity against lymphomas (Kohrt et al., 2014, Blood, 123: 678-86). Anti-KIR mAbs may be combined with ADCs, CAR-T or CAR-NK, interferons and/or checkpoint inhibitor antibodies to potentiate cytotoxicity to tumor cells or pathogenic organisms.

Antibody-Drug Conjugates

The subject CAR-T or CAR-NK constructs may be utilized in combination with one or more standard anti-cancer therapies, such as surgery, radiation therapy, chemotherapy and the like. In specific embodiments, the CAR-T or CAR-NK may be administered following use of a tumor debulking therapy, such as surgery, chemotherapy or immunotherapy. A preferred embodiment utilizes CAR-T or CAR-NK in combination with antibody-drug conjugates (ADCs).

ADCs are a potent class of therapeutic constructs that allow targeted delivery of cytotoxic agents to target cells, such as cancer cells. Because of the targeting function, these compounds show a much higher therapeutic index compared to the same systemically delivered agents. ADCs have been developed as intact antibodies or antibody fragments, such as scFvs. The antibody or fragment is linked to one or more copies of drug via a linker that is stable under physiological conditions, but that may be cleaved once inside the target cell. ADCs approved for therapeutic use include gemtuzumab ozogamicin for AML (subsequently withdrawn from the market), brentuximab vedotin for ALCL and Hodgkin lymphoma, and trastuzumab emtansine for HER2-positive metastatic breast cancer (Verma et al., 2012, N Engl J Med 367:1783-91; Bross et al., 2001, Clin Cancer Res 7:1490-96; Francisco et al., 2003, Blood 102:1458-65). Numerous other candidate ADCs are currently in clinical testing, such as inotuzumab ozogamicin (Pfizer), glembatumomab vedotin (Celldex Therapeutics), SAR3419 (Sanofi-Aventis), SAR56658 (Sanofi-Aventis), AMG-172 (Amgen), AMG-595 (Amgen), BAY-94-9343 (Bayer), BIIB015 (Biogen Idec), BT062 (Biotest), SGN-75 (Seattle Genetics), SGN-CD19A (Seattle Genetics), vorsetuzumab mafodotin (Seattle Genetics), ABT-414 (AbbVie), ASG-5ME (Agensys), ASG-22ME (Agensys), ASG-16M8F (Agensys), IMGN-529 (ImmunoGen), IMGN-853 (ImmunoGen), MDX-1203 (Medarex), MLN-0264 (Millenium), RG-7450 (Roche/Genentech), RG-7458 (Roche/Genentech), RG-7593 (Roche/Genentech), RG-7596 (Roche/Genentech), RG-7598 (Roche/Genentech), RG-7599 (Roche/Genentech), RG-7600 (Roche/Genentech), RG-7636 (Roche/Genentech), anti-PSMA ADC (Progenics), lorvotuzumab mertansine (ImmunoGen), milatuzumab-doxorubicin (Immunomedics), IMMU-130 (Immunomedics), IMMU-132 (Immunomedics) and antibody conjugates of pro-2-pyrrolinodoxorubicin. (See, e.g., Li et al., 2013, Drug Disc Ther 7:178-84; Firer & Gellerman, J Hematol Oncol 5:70; Beck et al., 2010, Discov Med 10:329-39; Mullard, 2013, Nature Rev Drug Discovery 12:329, U.S. Pat. Nos. 8,877,202; 9,095,628.) Because of the potential of ADCs to act as potent anti-cancer agents with reduced systemic toxicity, they may be used either alone or as an adjunct therapy to reduce tumor burden.

In certain embodiments, an ADC of use may be selected from the group consisting of IM MU-130 (hMN-14-SN-38), IM MU-132 (hRS7-SN-38), other antibody-SN-38 conjugates, or antibody conjugates of a prodrug form of 2-pyrrolinodoxorubicin (P2PDOX). (See, e.g., U.S. Pat. Nos. 7,999,083; 8,080,250; 8,741,300; 8,759,496; 8,999,344; 8,877,202 and 9,028,833)

The combinations of therapeutic agents can be formulated according to known methods to prepare pharmaceutically useful compositions, wherein the CAR-T, PMA and or other active ingredients is provided in a mixture with a pharmaceutically suitable excipient. Sterile phosphate-buffered saline is one example of a pharmaceutically suitable excipient. Other suitable excipients are well-known to those in the art. See, for example, Ansel et al., PHARMACEUTICAL DOSAGE FORMS AND DRUG DELIVERY SYSTEMS, 5th Edition (Lea & Febiger 1990), and Gennaro (ed.), REMINGTON'S PHARMACEUTICAL SCIENCES, 18th Edition (Mack Publishing Company 1990), and revised editions thereof

In each of the embodiments and aspects of the invention, the subject is a human, a non-human primate, bird, horse, cow, goat, sheep, a companion animal, such as a dog, cat or rodent, or other mammal.

Kits

Various embodiments may concern kits containing components suitable for treating or diagnosing diseased tissue in a patient. Exemplary kits may contain one or more CAR-Ts or CAR-NKs, PMAs, and other components as described herein.

A device capable of delivering the kit components through some a selected route of administration may be included. One type of device, for applications such as parenteral delivery, is a syringe that is used to inject the composition into the body of a subject. In certain embodiments, a therapeutic agent may be provided in the form of a prefilled syringe or autoinjection pen containing a sterile, liquid formulation or lyophilized preparation.

The kit components may be packaged together or separated into two or more containers. In some embodiments, the containers may be vials that contain sterile, lyophilized formulations of a composition that are suitable for reconstitution. A kit may also contain one or more buffers suitable for reconstitution and/or dilution of other reagents. Other containers that may be used include, but are not limited to, a pouch, tray, box, tube, or the like. Kit components may be packaged and maintained sterilely within the containers. Another component that can be included is instructions to a person using a kit for its use.

The present invention may be embodied in other specific forms without departing from its structures, methods, or other essential characteristics as broadly described herein and claimed hereinafter. The described embodiments are to be considered in all respects only as illustrative, and not restrictive. The scope of the invention is, therefore, indicated by the appended claims, rather than by the foregoing description. All changes that come within the meaning and range of equivalency of the claims are to be embraced within their scope.

### Definitions

Unless defined otherwise, all technical and scientific terms used herein have the same meaning as commonly understood by one of ordinary skill in the art to which this invention belongs. All publications mentioned herein are incorporated herein by reference for the purpose of describing and disclosing devices, compositions, formulations and methodologies that are described in the publications and which might be used in connection with the presently described invention.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

In the following description, numerous specific details are set forth to provide a more thorough understanding of the present invention. However, it will be apparent to one of skill in the art that the present invention may be practiced without one or more of these specific details. In other instances, well-known features and procedures well known to those skilled in the art have not been described in order to avoid obscuring the invention.

Although the present invention is described primarily with reference to specific embodiments, it is also envisioned that other embodiments will become apparent to those skilled in the art upon reading the present disclosure, and it is intended that such embodiments be contained within the present inventive methods.

The practice of the present invention may employ, unless otherwise indicated, conventional techniques and descriptions of organic chemistry, polymer technology, molecular biology (including recombinant techniques), cell biology, biochemistry, and immunology, which are within the skill of the art. Such conventional techniques include polymer array synthesis, hybridization, ligation, and detection of hybridization using a label. Specific illustrations of suitable techniques can be had by reference to the example herein below. However, other equivalent conventional procedures can, of course, also be used. Such conventional techniques and descriptions can be found in standard laboratory manuals such as Genome Analysis: A Laboratory Manual Series (Vols. I-IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press), Stryer, L. (1995) Biochemistry (4th Ed.) Freeman, N.Y., Gait, “Oligonucleotide Synthesis: A Practical Approach” 1984, IRL Press, London, Nelson and Cox (2000), Lehninger, Principles of Biochemistry 3rd Ed., W. H. Freeman Pub., New York, N.Y. and Berg et al. (2002) Biochemistry, 5th Ed., W. H. Freeman Pub., New York, N.Y.

Unless otherwise specified, “a” or “an” means “one or more.” The terms “and” and “or” may be used to mean either the conjunctive or disjunctive. That is, both terms should be understood as equivalent to “and/or” unless otherwise stated.

A “therapeutic agent” is an atom, molecule, or compound that is useful in the treatment of a disease. Examples of therapeutic agents include antibodies, antibody fragments, peptides, drugs, toxins, enzymes, nucleases, hormones, immunomodulators, antisense oligonucleotides, small interfering RNA (siRNA), chelators, boron compounds, photoactive agents, dyes, and radioisotopes.

An “antibody” as used herein refers to a full-length (i.e., naturally occurring or formed by normal immunoglobulin gene fragment recombinatorial processes) immunoglobulin molecule (e.g., an IgG antibody) or an immunologically active (i.e., specifically binding) portion of an immunoglobulin molecule, like an antibody fragment. An “antibody” includes monoclonal, polyclonal, bispecific, multi specific, murine, chimeric, humanized and human antibodies.

An “antibody fragment” is a portion of an intact antibody such as F(ab′)2, F(ab)2, Fab′, Fab, Fv, scFv, or dAb. Regardless of structure, an antibody fragment as used herein binds with the same antigen that is recognized by the full-length antibody. For example, antibody fragments include isolated fragments consisting of the variable regions, such as the “Fv” fragments consisting of the variable regions of the heavy and light chains or recombinant single chain polypeptide molecules in which light and heavy variable regions are connected by a peptide linker (“scFv proteins”). “Single-chain antibodies”, often abbreviated as “scFv” consist of a polypeptide chain that comprises both a VH and a VL domain which interact to form an antigen-binding site. The VH and VL domains are usually linked by a peptide of 1 to 25 amino acid residues. Antibody fragments also include diabodies, triabodies and single domain antibodies (dAb).

A “chimeric antibody” is a recombinant protein that contains the variable domains including the complementarity determining regions (CDRs) of an antibody derived from one species, preferably a rodent antibody, while the constant domains of the antibody molecule are derived from those of a human antibody. For veterinary applications, the constant domains of the chimeric antibody may be derived from that of other species, such as a cat or dog.

A “humanized antibody” is a recombinant protein in which the CDRs from an antibody from one species; e.g., a rodent antibody, are transferred from the heavy and light variable chains of the rodent antibody into human heavy and light variable domains, including human framework region (FR) sequences. The constant domains of the antibody molecule are derived from those of a human antibody. To maintain binding activity, a limited number of FR amino acid residues from the parent (e.g., murine) antibody may be substituted for the corresponding human FR residues.

A “human antibody” is an antibody obtained from transgenic mice that have been genetically engineered to produce specific human antibodies in response to antigenic challenge. In this technique, elements of the human heavy and light chain locus are introduced into strains of mice derived from embryonic stem cell lines that contain targeted disruptions of the endogenous heavy chain and light chain loci. The transgenic mice can synthesize human antibodies specific for human antigens, and the mice can be used to produce human antibody-secreting hybridomas. Methods for obtaining human antibodies from transgenic mice are described by Green et al., Nature Genet. 7:13 (1994), Lonberg et al., Nature 368:856 (1994), and Taylor et al., Int. Immun. 6:579 (1994). A human antibody also can be constructed by genetic or chromosomal transfection methods, as well as phage display technology, all of which are known in the art. (See, e.g., McCafferty et al., 1990, Nature 348:552-553 for the production of human antibodies and fragments thereof in vitro, from immunoglobulin variable domain gene repertoires from unimmunized donors). In this technique, antibody variable domain genes are cloned in-frame into either a major or minor coat protein gene of a filamentous bacteriophage, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. In this way, the phage mimics some of the properties of the B cell. Phage display can be performed in a variety of formats, for their review, see, e.g. Johnson and Chiswell, Current Opinion in Structural Biology 3:5564-571 (1993). Human antibodies may also be generated by in vitro activated B cells. (See, U.S. Pat. Nos. 5,567,610 and 5,229,275).

As used herein, the term “antibody fusion protein” is a recombinantly produced antigen-binding molecule in which an antibody or antibody fragment is linked to another protein or peptide, such as the same or different antibody or antibody fragment or another peptide or protein. The fusion protein may comprise a single antibody component, a multivalent or multispecific combination of different antibody components or multiple copies of the same antibody component. The fusion protein may additionally comprise an antibody or an antibody fragment and a therapeutic agent.

An antibody preparation, or a composition described herein, is said to be administered in a “therapeutically effective amount” if the amount administered is physiologically significant. An agent is physiologically significant if its presence results in a detectable change in the physiology of a recipient subject. In particular embodiments, an antibody preparation is physiologically significant if its presence invokes an antitumor response or mitigates the signs and symptoms of an infectious disease state. A physiologically significant effect could also be the evocation of a humoral and/or cellular immune response in the recipient subject leading to growth inhibition or death of target cells.

A “linking domain” or “linker” connects and spaces apart the epitope for binding to mCAR (e.g., ICG) and one or more antigen-binding moieties for binding to antigen(s) on the surface of a targeted cell (e.g., a tumor cell). The epitope for binding to mCAR and the antigen-binding moiety(-ies) can be directly conjugated by standard techniques, in which case the PMA has no linking domain. However, the use of a linking domain to connect the two molecules can be helpful as it can provide flexibility and stability to the PMA depending on the identity of the components comprising the PMA. Examples of suitable linking domains include: (1) polyethylene glycol (PEG); (2) polyproline; (3) hydrophilic amino acids; (4) sugars; (5) unnatural peptideoglycans; (6) polyvinylpyrrolidone; (7) pluronic F-127. Linkers lengths that are suitable include, but are not limited to, linkers having 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40, or more atoms.

As used herein, the terms “affinity agent” refers to (1) a portion of a mCAR that specifically binds to the ICG moiety of a PMA (or other recognition moiety), or (2) a portion of a PMA that specifically binds the a tumor cell surface antigen (or more generally a cell-surface antigen of any targeted cell). The affinity agent and the molecule to which it binds specifically constitutes a specific binding pair. The binding between the members of the binding pair is generally noncovalent, although a covalent (e.g., disulfide) linkage between binding pair members can also be used. Exemplary binding pairs include, but are not limited to: (a) a haptenic or antigenic compound in combination with a corresponding antibody, or binding portion or fragment thereof; (b) a nucleic acid aptamer and protein; (c) nonimmunological binding pairs (e.g., biotin-avidin, biotin-streptavidin, biotin-Neutravidin, biotin-Tamavidin, streptavidin binding peptide-streptavidin, glutathione-glutathione S-transferase); (d) hormone-hormone binding protein; (e) receptor-receptor agonist or antagonist; (f) lectin-carbohydrate; (g) enzyme-enzyme cofactor; (h) enzyme-enzyme inhibitor; (i) complementary oligonucleotide or polynucleotide pairs capable of forming nucleic acid duplexes; (j) thio (—S—) or thiol (—SH) containing binding member pairs capable of forming an intramolecular disulfide bond; and (k) complementary metal chelating groups and a metal (e.g., metal chelated by the binding pairs nitrilotriacetate (NTA) and a 6x-His tag). Specific binding pair members need not be limited to pairs of single molecules. For example, a single ligand can be bound by the coordinated action of two or more antiligands.

In the context of the binding of an affinity agent, the terms “specific binding,” “specifically binds,” and the like refer to the preferential association of an affinity agent with a targeted molecule, in comparison to a control molecule. Specific binding of an affinity agent generally means an affinity of at least 10-6 M⁻¹ (i.e., an affinity having a lower numerical value than 10⁻⁶ M⁻¹ as measured by the dissociation constant Kd). Affinities greater than 10⁻⁸ M⁻¹ are preferred. Specific binding can be determined using any assay for binding known in the art, including, for antibodies and antibody fragments, Western Blot, enzyme-linked immunosorbent assay (ELISA), flow cytometry, and immunohistochemistry.

### EXAMPLES

### Example 1. Generation of Tandem CD19-CD22 PMA and CD22-CD19 PMA

Bi-specific CD19-CD22 PMAs are constructed by isothermal assembly (Gibson et al., Nat Methods 6:343-345, 2009) of DNA fragments encoding the CD19 single-chain variable fragment (scFv) derived from human mAb clone FMC63 (Nicholson et al., Mol Immunol 34:1157-1165, 1997) and the CD22 scFv derived from human CD22 mAb clone M971 (Xiao et al., MAbs 1:297-303, 2009). The scFv regions of CD19 and CD22 are linked to each other sequentially by a flexible interchain linker consisting of five repeats of four glycines followed by one serine ((G4S1)5). It has been reported that the different positions of these two scFv in tandem adaptor show different binding efficacy (Schneider et al., J Immunotherapy Cancer 5:42-59, 2017). Therefore we use the better-performing of two formats of PMA: CD19-CD22 tandem PMA (1922PMA) and CD22-CD19 tandem PMA (2219PMA). The tandem scFVs are expressed in Esherichia coli (E. coli) and purified as previously described (Feldmann et al., J Immunol 189:3249-3259, 2012).

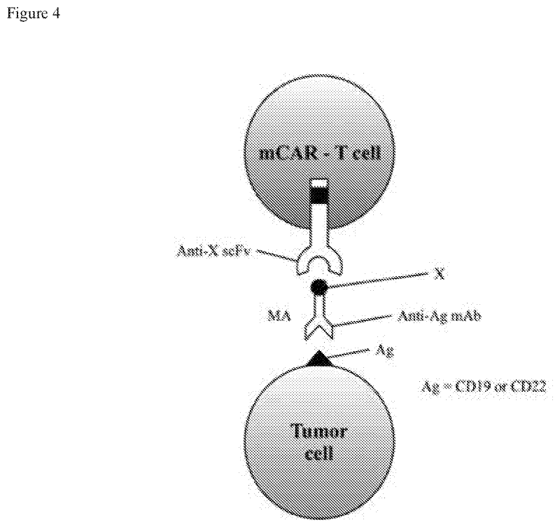

To compare the tandem PMAs, a set of monospecific MAs, including CD19 MA and CD22 MA, are also prepared. An example of a monospecific MA-mCAR-T system is shown in FIG. 4.

### Example 2: Conjugation of ICG to the PMAs

The tandem PMA is reacted in conjugation buffer with the amine-reactive ICG-N-hydroxysulfosuccinimide ester (abbreviated as ICG-sulfo-OSu) dissolved in anhydrous DMSO, at ICG:PMA ratio 5:1. Then the mixture is followed by doubly purified by SE-HPLC and ethyl acetate extraction to remove impurities and non-covalent ICG (Yang et al., Bioconjugate Chem 25:1801-1810, 2014). The ICG conjugation site and valency is selected to optimize anti-ICG CAR-T activity, since it has been reported that the site and stoichiometry of conjugation of FITC to anti-CD19 Fab affects anti-FITC CAR-T activity (Ma et al., Proc Natl Acad Sci USA, 113:E450-E458, 2016).

### Example 3: Construction of mCAR-T Cells and Preparation of Target Cells

mCAR construction, as well as T cell selection, activation and expansion, is conducted as previously described (Milone et al., Molecular Therapy 17:1453-1464, 2009; Levine et al., Proc Natl Acad Sci USA 103:17372-17377, 2006; Song et al., Cancer Res 71:4617-4627, 2011). To create a mCAR that recognizes the PMAs, the scFv developed in Example 1 is incorporated into a second generation CAR construct harboring the human CD8 hinge (spacer), CD8 transmembrane, 4-1BB costimulatory domain, and CD3 activation domains (FIG. 2). This design is similar to the second generation CAR used by June and coworkers in CART-19 (Milone et al., Molecular Therapy 17:1453-1464, 2009).

The CAR constructs are cloned into a pCDH lentiviral (LV) vector (System Biosciences, Palo Alto, Calif.). There are two major advantages for a pCDH LV vector: (1) it is a third generation/self-inactivating (SIN) LV vector. The 3′ LTR of the vector is modified, with tat being eliminated and rev provided in a separate plasmid; (2) it contains a 2A peptide for co-expression of a reporter gene. The 2A-like sequence (T2A) from the insect virus Thosea asigna mediates the co-expression of a reporter gene with the target cDNA.

Human CD3+ T cells are obtained by Ficoll-Pacque purification of peripheral blood monocytes (PBMCs) from healthy donor whole blood (Cureline Inc., South San Francisco, Calif.). Efficiencies of 50-75% are expected for lentiviral transduction of this mCAR construct into CD3 T cells from freshly isolated human PBMCs, which is comparable to the FMC63-based CART-19.

The leukemia cell lines NALM-6 and K562 are used as target cells. CD19+, CD22+, and CD19+/CD22+K562 cells are generated by lentiviral transducing parental K562 cells with CD19 and/or CD22 constructs (Table 1). For comparison of the CD19-CD22 tandem PMA-mCAR system with in vitro and in vivo assays, conventional second generation single CARs are used as positive controls: CD19 CAR and CD22 CAR.

TABLE 1
Cell lines used for in vitro and in vivo assays

| | Cell Line | | | |
|---|---|---|---|---|
| Cell Line | Description | Phenotype | Source | Note |
| NALM-6 | Acute | CD19+, CD22+, | Imanis Life | Firefly luciferase |
| | lymphocytic | Fluc+ | Sciences | introduced by LV |
| | leukemia (ALL) | | | |
| | cell lines | | | |
| K562 | Chronic | CD19−, CD22− | ATCC | Parental line |
| | myelogenous | | | |
| | leukemia line | | | |
| | | CD19+, CD22− | Prepared | CD19 introduced |
| | | | in house | by LV |
| | | CD19−, CD22+ | Prepared | CD22 introduced |
| | | | in house | by LV |
| | | CD19+, CD22+ | Prepared | CD19 and CD22 |
| | | | in house | introduced by LV |

### Example 4. Evaluation of the Tandem PMA-mCAR-T System In Vitro and In Vivo

Activities of the tandem CARs for PMA-dependent target cell lysis are evaluated in vitro with cytotoxicity assays against leukemia cell lines NALM-6 and K562 cells. Therapeutic function of the top candidates of the dual CARs are then validated in vivo against these NALM6 leukemia lines. Some of these dual CARs are further tested against patient-derived xenografts.

In Vitro Assays

mCAR protein surface expression in transduced T cells is validated by FACS analysis. A cytokine releasing assay (Kalos et al., Sci Transl Med. 3: 95ra73, 2011) is used to test specific antigen recognition by the PMA-mCAR-T system through measuring the releasing level of IFN-γ and IL-2 by ELISA kits.

To determine the optimal tandem PMA design for CD19 and CD22, the ability of the PMA-mCAR-T cells to lyse tumor target cells is measured by a cytotoxicity assay (Kalos et al., Sci Transl Med. 3: 95ra73, 2011). Tumor target cells such as NALM-6 (CD19+CD22+) and K562 cells with different CD19 and CD22 expression phenotypes are treated by different types of PMA-mCAR-T system and different concentrations to test their potency.

(2) In Vivo Assays

In vivo analysis of PMA-mCAR activity is conducted using a xenograft model with NOD-SCID r-chain deficient (NSG) mice. 6-8 week-old female NSG mice are inoculated with 5×10⁵ luciferized Nalm-6 (CD19+CD22+) intravenously (day 0), and engraftment is confirmed by bioluminescence imaging (Fry et al., Nature Medicine doi:10.1038/nm.4441, 2017). On day 6, 40×10⁶ CAR-T cells (50-75% CAR+) are infused intravenously, and adaptors are dosed 6 hours later. In parallel, control groups (tumor-bearing mice that receive no treatment, mCART only, single CD19 CAR T cells, single CD22 CAR-T cells) are injected with PBS. Body weight is monitored daily, and tumor growth is monitored weekly by bioluminescence imaging. There are two independent experiments, 6-8 mice per group.

Although the foregoing invention has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference. Where a conflict exists between the instant application and a reference provided herein, the instant application shall dominate.

1. A precision molecular adaptor (PMA) comprising: (a) a recognition moiety comprising a member of the group consisting of indocyanine green (ICG), an ICG derivative, methylene blue, and a methylene blue derivative, and, attached to the recognition moiety, (b) an antigen-binding moiety comprising a first antigen recognition domain that binds specifically to a first target antigen, and optionally wherein the binding moiety further comprises a second antigen recognition domain that binds specifically to a second target antigen, wherein the second target antigen is different than the first target antigen.

2. (canceled)

3. The PMA of claim 1, wherein the first target antigen and the second target antigen are cell surface proteins or cell surface protein complexes, and/or wherein the first target antigen and the second target antigen are selected from the group consisting of: alpha-fetoprotein, α-actinin-4, A3, antigen specific for A33 antibody, ART-4, B7, Ba 733, BAGE, BrE3-antigen, a member of the carbohydrate antigen family, a member of the carbonic anhydrase family, CA125, CAMEL, CAP-1, CASP-8/m, CCL19, CCL21, CD1, CD1a, CD2, CD3, CD4, CD5, CD8, CD10, CD11A, CD14, CD15, CD16, CD18, CD19, CD20, CD21, CD22, CD23, CD25, CD29, CD30, CD32b, CD33, CD37, CD38, CD40, CD40L, CD44, CD45, CD46, CD52, CD54, CD55, CD59, CD64, CD66a-e, CD67, CD70, CD70L, CD74, CD79a, CD79b, CD80, CD83, CD95, CD99, CD 123, CD126, CD132, CD133, CD138, CD147, CD154, CD274, CDC27, CDK-4/m, CDKN2A, CTLA4, CXCR4, CXCR7, CXCL12, HIF-1α, colon-specific antigen-p, CEACAM-5, CEACAM-6, c-Met, DAM, a diasialoganglioside, an embryonic antigen, a member of the epidermal growth factor receptor family and mutants thereof, a member of the ephithelia glycoprotein family, EGP-1, EGP-2, ELF2-M, Ep-CAM, a member of the ephrin receptor family, erb-1, erb-2, fibroblast growth factor, Flt-1, Flt-3, folate binding protein, α-folate receptor, follicle stimulating hormone receptor, G250 antigen, GAGE, gp100, a member of the Rho family of GTPases, GRO-β, HLA-DR, HM1.24, human chorionic gonadotropin and its subunits, HER2/neu, a member of the high mobility group proteins and mutants thereof, HMGB-1, human high molecular weight-melanoma-associated antigen, hypoxia inducible factor, HSP70-2M, HST-2, Ia, IGF-1R, IFN-γ, IFN-α, IFN-β, IFN-k, IL-4R, IL-6R, IL-13R, IL-15R, IL-17R, IL-18R, IL-2, IL-6, IL-8, IL-12, IL-15, IL-17, IL-18, IL-23, IL-25, insulin-like growth factor-1, KC4-antigen, KS-1-antigen, KS1-4, Le-Y, LDR/FUT, a ligand of the NKG2D receptor, macrophage migration inhibitory factor, MAGE, MAGE-3, MART-1, MART-2, NY-ESO-1, TRAG-3, mCRP, MCP-1, MIP-1A, MIP-1B, MIF, a members of the mucin protein family, MUM-1/2, MUM-3, NCA66, NCA95, NCA90, pancreatic cancer mucin, PD1 receptor, placental growth factor, p53, PLAGL2, prostatic acid phosphatase, a prostrate-specific antigen, PRAME, P1GF, ILGF, ILGF-R, IL-6, IL-25, RS5, RANTES, T101, SAGE, 5100, survivin, survivin-2B, TAC, TAG-72, tenascin, TIM-3, a TRAIL receptor, TNF-α, Tn antigen, a Thomson-Friedenreich antigen, a tumor necrosis antigen, a members of the vascular endothelial growth factor receptor family, ED-B fibronectin, WT-1, 17-1A-antigen, complement factor C3, complement factor C3a, complement factor C3b, complement factor C5a, complement factor C5, an angiogenesis marker, bcl-2, bcl-6, Kras, an oncogene marker and an oncogene product.

4-6. (canceled)

7. A method of making the PMA of claim 1 comprising:
providing an antigen-binding moiety comprising a first antigen recognition domain that binds specifically to a first target antigen, and
attaching to the antigen-binding moiety a recognition moiety comprising an member of the group consisting of indocyanine green (ICG) moiety, an ICG derivative, methylene blue, and a methylene blue derivative.

8. A chimeric antigen receptor (CAR) comprising:
(a) a binding domain that specifically binds to a recognition moiety comprising an member of the group consisting of indocyanine green (ICG) moiety, an ICG derivative, methylene blue, and a methylene blue derivative;
(b) an extracellular hinge and transmembrane domain; and
(c) a signal transduction domain.

9. The CAR of claim 8 wherein the binding domain comprises an antibody or antibody fragment that specifically binds to the recognition moiety.

10. The CAR of claim 9 wherein said antibody or antibody fragment is an scFv.

11. The CAR of claim 8 wherein the signal transduction domain comprises a T cell activation signal region.

12. The CAR of claim 11 wherein the signal transduction domain further comprises one or more costimulatory signal regions.

13. A polynucleotide construct comprising a promoter operably linked to a sequence that encodes the CAR of claim 8.

14. An effector cell comprising the polynucleotide construct of claim 13.

15. An effector cell of claim 14 selected from the group consisting of: a T cell, a Natural Killer cell, a cytotoxic T lymphocyte, a regulatory T cell, a neutrophil, a macrophage, an rδ T cell, a Natural Killer T cell, and a lymphokine-activated killer cell.

16. (canceled)

17. An allogeneic cell of claim 14.

18. A two component therapeutic comprising:
a composition comprising a plurality of effector cells that comprise a vector comprising a eukaryotic promoter operably linked to a sequence that encodes the CAR of claim 8; and
a composition comprising a precision molecular adaptor (PMA), the PMA comprising: (a) the recognition moiety, and, attached to the recognition moiety, (b) an antigen-binding moiety comprising a first antigen recognition domain that binds specifically to a first target antigen.

19. A method of treating a mammal having a disease comprising:
introducing into the mammal a therapeutically effective amount of a composition comprising one or more effector cells that comprise a vector comprising a eukaryotic promoter operably linked to a sequence that encodes the CAR of claim 8; and
introducing into the mammal a therapeutically effective amount of a composition comprising a precision molecular adaptor (PMA), the PMA comprising: (a) the recognition moiety, and, attached to the recognition moiety, (b) an antigen-binding moiety comprising a first antigen recognition domain that binds specifically to a first target antigen, thereby reducing a symptom of the disease.

20. The method of claim 19 wherein said one or more effector cells are selected from the group consisting of: a T cell, a Natural Killer cell, a cytotoxic T lymphocyte, a regulatory T cell, a neutrophil, a macrophage, an rδ T cell, a Natural Killer T cell, and a lymphokine-activated killer cell.

21. (canceled)

22. The method of claim 19 further comprising introducing into one or more effector cells from the mammal said vector, thereby producing said plurality of cells that comprise said vector.

23. The method of claim 19 further comprising:
isolating said effector cells from the mammal; and
introducing said vector into said effector cells isolated from the mammal.

24. (canceled)

25. A method of identifying locations of cells comprising a CAR, the method comprising:
introducing into the mammal a therapeutically effective amount of a composition comprising one or more effector cells that comprise a vector comprising a eukaryotic promoter operably linked to a sequence that encodes a chimeric antigen receptor (CAR), wherein the CAR comprises: (a) a binding domain that specifically binds to a recognition moiety comprising an member of the group consisting of indocyanine green (ICG) moiety, an ICG derivative, methylene blue, and a methylene blue derivative; (b) an extracellular hinge and transmembrane domain; and (c) a signal transduction domain;
introducing into the mammal a therapeutically effective amount of a composition comprising a precision molecular adaptor (PMA), the PMA comprising: (a) the recognition moiety, and, attached to the recognition moiety, (b) an antigen-binding moiety comprising a first antigen recognition domain that binds specifically to a first target antigen; and
performing near infrared fluorescent imaging to identify the location of the effector cells.

26. A kit for producing the PMA of claim 1, the kit comprising:
a first component comprising a member of the group consisting of indocyanine green (ICG) moiety, an ICG derivative, methylene blue, and a methylene blue derivative,
a second component comprising an antigen-binding moiety comprising a first antigen recognition domain that binds specifically to a first target antigen, and
a container for said first and second components,
wherein said PMA comprises said first component attached to said second component.

27. A kit comprising:
a first component selected from the group consisting of: a vector comprising a eukaryotic promoter operably linked to a sequence that encodes the CAR of claim 8; and
a second component consisting of a composition comprising a precision molecular adaptor (PMA), the PMA comprising a recognition moiety comprising the recognition moiety, and, attached to the recognition moiety, an antigen-binding moiety comprising a first antigen recognition domain that binds specifically to a first target antigen; and
a container for said first and second components.
